(19) United States
(12) Patent Application Publication
(10) Pub. No.: US 2005/0272103 A1
(43) Pub. Date: Dec. 8, 2005

(54) Cell separation matrix

(76) Inventor: Wen-Tien Chen, Stony Brook, NY (US)

Correspondence Address: PILLSBURY WINTHROP SHAW PITTMAN, LLP, P.O. BOX 10500, MCLEAN, VA, 22102, US

(21) Appl. No.: 11/175,287

(22) Filed: Jul. 7, 2005

Related U.S. Application Data
(63) Continuation of application No. 10/122,268, filed on Apr. 11, 2002, now abandoned.
(63) Continuation-in-part of application No. PCT/US01/26735, filed on Aug. 28, 2001.
(60) Provisional application No. 60/332,408, filed on Nov. 16, 2001.
(60) Provisional application No. 60/231,517, filed on Sep. 9, 2000.

(51) Int. Cl. G01N 33/574; C12M 1/34

(52) U.S. Cl. 435/7.23; 435/287.2

(57) ABSTRACT

A novel modified matrix system, mimicking a metastatic environment, that can be used to capture and detect viable cancer and normal cells from tissue fluid samples derived from cancer subjects and which provides effective cell separation for diagnostic and therapeutic applications in treating patients with metastatic diseases.

### RELATED APPLICATIONS

This application claims priority from U.S. Provisional Patent Application No. 60/332,408, filed Nov. 16, 2001, and is a continuation-in-part application of parent application PCT/US01/26735, filed Aug. 28, 2001, and U.S. Provisional Patent Application No. 60/231,517, filed Sep. 9, 2000.

### BACKGROUND OF THE INVENTION

1. Field of the Invention

The present invention generally relates to a matrix for separating cells. More particularly, the present invention relates to a cell-separation matrix that may be used to selectively isolate cells with metastatic potential. The cell-separation matrix may be used in the diagnosis of metastatic cancers and in the treatment of cancer by reducing circulating metastatic cells.

2. Description of the Related Art

Primary cancers frequently shed neoplastic cells into the circulation at an early stage of metastases formation (Fidler I J, 1973, European Journal of Cancer 9:223-227; Liotta L A et al., 1974, Cancer Research 34:9971004). Patients with metastatic disease may release large numbers of cancer cells into the circulation, in many cases approaching release-rates of 10⁷ to 10⁹ cells per day (Glaves, D., R P Huben, & L. Weiss. 1988. Br. J. Cancer. 57:32-35). However, studies suggest that only a minor subpopulation of shed cancer cells, ranging from one of thousands to millions of cells, are metastatic (Glaves, D., 1983, Br. J. Cancer, 48:665-673). The fact is that the majority of shed cancer cells do not survive in the circulation (Weiss and Glaves, 1983; Karczewski et al., 1994). Experimental data suggest that the initial release of cancer cells from a primary tumor is not the limiting factor in metastatic development. When tumor cells are introduced directly into the circulation of mice or rats, less than 0.01% of such cells form tumor nodules. More commonly the efficiency is two or more orders of magnitude lower (Luzzi, K. J. et al. 1998. Am. J. Pathol. 153, 865-873).

It has been suggested that the adhesion of metastatic cells to the extracellular matrix of basement membrane and connective tissue underlying vessel walls and subsequent tissue degradation are key events for metastases formation in an organ (Liotta et al., 1991, Cell 64:327-336). It is also believed that angiogenesis, that is the process of signaling new blood vessel growth into a growing tumor mass, is required for the survival, growth and metastasis of cancer cells (Folkman, 1995). It is known that there a small number of endothelial cell progenitors or angioblasts circulate in human peripheral blood (Asahara et al., 1997). In addition, it is known that a small percentage of leukocytes in human peripheral blood that are activated to associate with circulating cancer cells. It is possible that during intravascular metastases formation, a small fraction of circulating cancer cells, as well as hematopoietic cells comprising endothelial cell progenitors and cancer cell-associating leukocytes, preferentially attach to sites where connective tissue structure has been modified due to local wound or inflammatory responses. The modified matrix may allow local invasion and growth of solitary cells (Clark et al., 1985)(AI-Mehdi, A. B. et al., 2000, Nature Medicine. 6, 100-102).

The present inventor has hypothesized that it would be useful both for diagnostic and therapeutic purposes to separate the small fraction of circulating cancer cells that are metastatic, as well as the rare endothelial cell progenitors and cancer cell-associating leukocytes, from the large number of other circulating cells in a patient's body. Two major problems have been identified with respect to such cancer cell separation proposal: (1) the proposed method must isolate specifically viable cancer and related tissue cells but leave alone unrelated or damaged cells (Karczewski et al., 1994), and (2) that the proposed method must achieve the specificity in cell separation of one cell from over one million nucleate cells, or over one billion cells in whole blood. There are approximately 10⁹ red cells and 10⁷ white nucleate cells present in one cubic centimeter (c.c.) or gram of blood. It is estimated that among the order of 10 billion total mononuclear cells harvested from a patient with metastatic cancer, there are 25 thousand to 12 million contaminating cancer cells during traditional bone marrow harvest and leucopheresis procedures (Campana, D. et al. 1995, Blood 85:1416-34)(Brugger et al., 1999; Brugger et al., 1994; Brugger et al., 1995). These contaminating cancer cells have been shown by genetic marking to contribute to relapse (Rill, E R et al., 1994, Blood 84:380-383). Because of the danger associated with such cells, there exists a great need for efficient methods for removing viable cancer cells from a hematopoietic cell transplant (Gulati, S C et al. 1993, Journal of Hematotherapy, 2:467-71).

Several methods are known for detecting cancer cells from background tissue cells. Traditional diagnosis utilizes the different morphology of tumor cells, as compared to normal cells of the blood and normal tissue cells, followed by immunocytochemistry using developmental lineage tissue markers such as antibodies against hematopoietic and epithelial cells. For example, immuno-morphologic analysis may be performed by cytospin preparations or smears of marrow, peripheral blood or lymph node cell samples, followed by May Grunwald-Giemsa staining or stained with tissue specific antibodies, and examination by light microscopy (Molino et al., 1991. Cancer, 67:1033). Alternatively, rare circulating cancer cells have also been detected through the use of sensitive, reverse transcriptase polymerase chain reaction (RT-PCR) to amplify putative tumor markers or epithelial markers such as prostate specific antigen (PSA) mRNA or cytokeratin 19 mRNA (Peck et al., 1998; Wang et al., 2000).

Microdissection methods are known for separating rare cancer cells from major tissue cells one by one (Suarez-Quian et al., 1999, Biotechniques, 26:328-35; Beltinger and Debatin, 1998, Mol. Pathol 51:233-6). These methods have several disadvantages, particularly with respect to complicated sample processing, no reference for cell viability, and false-positive results. Alternative approaches to cell separation are based on physical characteristics of tumor cells such as shape, size, density or electrical charge (Vona et al., 2000). Circulating nucleated cells can be readily separated from large number of background red blood cells as a group called “buffy coat” on density gradients by centrifugation (Dicke et al., 1970, Exp. Hematol. 20:126-130; Olofsson et al., 1980, Second J. Hematol. 24:254-262; Ellis et al., 1984, J. of Immunological Methods 66:9-16; Sabile et al., 1999, Am. J. Clin. Pathol. 112:171-8). However, such methods are dependent on the availability of the buoyant density and morphology unique to different nucleated cells, and various cancer cells seem to have different physical characteristics.

Most recent approaches to cell separation are antibody-based. Immuno-affinity methods involve affixing an antibody on a carrier or fluorescent label, in which antibody reacts to an antigenic epitope present on the surface of the cells of interest. The methods include affinity chromatography, immuno-precipitation, and flow cytometry or called fluorescence activated cell sorting (FACS). Flow cytometry separates and detects individual cells one-by-one from a large number of background cells (Herzenberg et al., 1979, Proc. Natl. Aca. Sci. USA 76: 1453-5; Pituch-Noworolska et al., 1998, Int. J. Mol. Med. 1:573-8). It has been shown that breast carcinoma cells can be isolated and identified from a peripheral blood sample by flow cytometry (Gross et al., 1995. Proc. Natl. Aca. Sci. USA. 92:537). However, it could not resolve cells that existed in clusters, which may be the case in some cancers.

Other popular antibody-based, cell sorting approaches involve separating cancer cells from a large number of background cells using antibody-coated microbeads in a centrifugation or filtration process (Dicke et al., 1968, Transplantation 6:562-570). The antibody-coated microbeads may comprise a magnetic material to permit separation of the cancer cell-bound antibody-coated microbeads from a challenge solution by way of a magnetic field (Shpall et al., 1991, Bone Marrow Transplantation 7:145-151; Durrant et al., 1992, J. Immunol. Meth. 147:57-64; Denis et al., 1997, Int. J. Cancer 74:540-4; Racila et al., 1998, Proc. Natl. Acad Sci USA 95-4589-94).

There are numerous disadvantages associated with antibody-based cell separation methods, including flow cytometry and magnetic cell separation. For one, cancer cells often variably express tumor- or tissue specific antigens (Sabile et al., 1999). There is also frequently significant non-specific antibody binding to damaged cells, with such techniques often including no reference for cell viability. Overall such antibody-based cell separation methods have a higher than desired false-positive rate. Furthermore, these cell separation methods are time consuming and cost intensive.

In co-pending International PCT Application No. PCT/US01/26735, filed Aug. 28, 2001, claiming priority to U.S. Provisional Patent Application No. 60/231,517, there is described a fibrous matrix scaffolding coated with blood-borne adhesion molecules, such as human plasma fibronectin, laminin and vitronectin, which supports the attachment of cancer cells and may be used to isolate metastatic cells from other cells. The fibrous matrix scaffolding of such application may be made of a number of materials including collagenous fibers, fibrin gels, purified cotton or plastic fibers. The matrix may be housed in a vessel. The cells captured by the matrix are assayed ex vivo as putative metastatic cells: (1) for their viability by apoptosis and cytotoxicity assays, (2) for their cell proliferation, and (3) for measurement of their metastatic potential, i.e., assaying their ability to digest and internalize matrix fragments, simultaneously. In addition, conventional pathological methods for detecting cancer cells may be used, including cell size, nuclear shape, and immunocytochemical reactivity against tissue markers, such as PSA, cytokeratins, pan-epithelial antigen BerEP4 present on normal and neoplastic epithelial cells. The co-pending patent application is based on the observation that cancer cells present in the circulation of patients with metastatic diseases can attach to tissue fragments and form large cellular clusters. This observation suggests that natural structural scaffolds promote attachment of metastasized cancer cells, as well as hematopoietic cells associated with metastasis. Co-pending International PCT Application No. PCT/US01/26735 discloses that type I/III collagen, fibrin, purified cotton, and mechanically scratched surfaces of tissue culture plastic, absorb preferentially blood-borne adhesion components that promote adhesion of cancer cells.

Also described in co-pending International PCT Application No. PCT/US01/26735 is a method for inhibiting the metastatic potential of cancer cells by administration of modulators of serine integral membrane proteases, in particular those inhibitors that interfere in the formation of a protease complex comprising seprase and dipeptidyl peptidase IV (“DPPIV”).

Several cell separation systems are presently available for separation of circulating cancer cells from blood of cancer patients. Table 1 summarizes some of the characteristics of the available methodologies, including a density gradient centrifugation separating cells by cell density, a filtration based on cell or clump size, flow cytometry or microscopy of fluorescent antibody-targeted cells, magnetic separation using cells bound by antibody-magnetic particles, and a functional separation for viable cells based upon a matrix described in International PCT Application PCT/US01/26735 (filed by the present inventor).

TABLE 1
Human circulating cancer cells resolved by different methods

| | | | Cell | |
|---|---|---|---|---|
| Methods | Cells/mL* | Emboli/mL** | viability | References |
| (1) Antibody-antigen reaction | 14-21,209 | 3-1,462 | Not known | Glaves et al., |
| followed by centrifugation | | | | 1988 |
| (2) Negative antibody | 2-5 | 2-5 | Not known | Tśo et al., 1977; |
| depletion followed by | | | | Wang et al., 2000 |
| centrifugation | | | | |
| (3) Autotransfusion followed | 1032-101,025 | 56-8,370 | Mostly | Karczewski et al., |
| by filtration by cell size | | | dead | 1994 |
| (4) Filtration by cell size | 1-3 | 1-12 | Not known | Vona et al., 2000 |
| (5) Antibody-fluorescence | 3,710-10,200 | Not known | Not known | Kraeft et al., 2000 |
| microscopic imaging | | | | |
| (6) Antibody-magnetic | 2-6 | Not known | Not known | Racila et al., 1998 |
| fluid/flow cytometry | | | | |
| (7) Antibody-magnetic | 2-6 | Not known | Not known | Beitsch and |
| fluid/flow cytometry | | | | Clifford, 2000 |
| (8) Functional affinity to | 182-18,003 | 3-1,231 | Viable | Co-pending |
| matrix of International PCT | | | | International PCT |
| Application PCT/US01/26735 | | | | Application |
| | | | | PCT/US01/26735 |
| | | | | claiming priority |
| | | | | to U.S. Provisional |
| | | | | Patent Applic. No. |
| | | | | 60/231,517 |

*Range of putative cancer cells found in one milliliter of blood or in 10⁶ equivalent nucleated blood cells by particular methods, which have demonstrated the sensitivity of 1 cell per mL and background level (no or few cells) in the blood from normal donor.
**Range of cell dusters or clumps containing 5-100 putative cancer cells found in one milliliter of blood or equivalent nucleated blood cells by particular methods, which have demonstrated the sensitivity of 1 cell per mL and background level (no or few cells) of blood from normal donor.

### SUMMARY OF THE INVENTION

The present invention provides a cell-separation matrix modified from that described in co-pending PCT Patent Application PCT/US01/26735 (claiming priority to U.S. Provisional Patent Application No. 60/231,517) which provides an improved matrix for separating cells in a manner to isolate and detect metastatic cells, and a small fraction of hematopoietic cells associated with metastasis, from blood and tissues of patients inflicted with metastasic cancer. The modified-matrix provides a “cancer cell trap” that allows for the efficient removal of viable cancer cells from the tissue fluids. The modified-matrix is useful for separating over 99% of blood cells from such metastatic, and associated-metastatic, cells. Metastatic cells may be characterized by in vitro assays including the local collagen or fibronectin degradation and internalization, cell proliferation, pathological and immunocytochemical identification, and apoptotic and cytolytic assays.

The modified-matrix of the present invention utilizes an intermediate coating about a core material to effectuate improved absorption of blood-borne adhesion components that promote the adhesion of cancer cells. The intermediate coating comprises materials, including, but not limited to, gelatin, collagens, fibrin, proteoglycans, hyaluronate, and dextran, that has the affinity, or efficiently binds, to another material having the affinity, to bind blood-borne adhesion components that promote the adhesion of cancer cells, such as fibronectin, fibrin, heparin, laminin, tenascin or vitronectin, and synthetic compounds, such as synthetic fibronectin and laminin peptides and the like, and that has the ability to effectively coat the core material used in the matrix. For example, glutaraldehyde can be used to coat bone substrata and to bind blood-borne adhesion components that promote the adhesion of cancer cells. Gelatin has been found to be useful to coat core materials such as whole, denatured, polymers and fragments of bone, connective tissues (such as collagens, proteoglycans and hyaluronate), glass, inert polymeric materials (such as magnetic colloid, polystyrene, polyamide material like nylon, polyester materials cellulose ethers and esters like cellulose acetate, urethane foam material, DEAE-dextran), as well as other natural and synthetic materials, such as other foam particles, cotton, wool, dacron, rayon, acrylates and the like. The gelatin-coated core materials may then be crosslinked, for example, with glutaraldehyde, washed and the glutaraldehyde cross-linked, gelatin-coated, core material exposed to one or more blood-borne adhesion components that promote the adhesion of cancer cells. The blood-borne adhesion components that promote adhesion of cancer cells, may comprise fibronectin, fibrin, laminin, heparin, and vitronectin, or biological mimics thereof, and may be prepared by purification from natural sources or synthesized by artificial means.

The modified-matrix system of the present invention more efficiently captures and detects viable cancer and hematopoietic cells from tissue fluid samples derived from cancer subjects than that described in co-pending application PCT Patent Application PCT/US01/26735 (claiming priority to U.S. Provisional Patent Application No. 60/231,517). The modified matrix of the invention has affinity for metastatic cancer cells and a small fraction of hematopoietic cells, and it mimics the site at the vessel wall of arteriovenous anastomosis and loci of metastases, where extracellular matrix (ECM) components, including bone matrix, collagens, proteoglycans, fibronectin, laminin, fibrin, heparin, tenascin and vitronectin etc., have been modified during the process of intravasation. In essence, the modified matrix mimics a metastatic environment capturing cancer cells. The cancer cells isolated by the methods of this invention are viable, grow ex vivo, and exhibit the invasive activity against the ECM, i.e., partially degrading it followed by ingestion of ECM fragments by the cells.

In certain embodiments, bone fragments are used themselves as the core material, which form shapes of planar substrata or beads. While the bone substrata can be used directly to bind the blood-borne adhesion components that promote the adhesion of cancer cells, such substrata is more efficiently crosslinked with glutaraldehyde, followed by blocking with blood-borne adhesion components that promote the adhesion of cancer cells, for example, with 0.01-0.5 milligram per milliliter of human plasma fibronectin, fibrin, heparin, laminin, tenascin, vitronectin, or synthetic compounds, such as synthetic fibronectin and laminin peptides and the like. The coated-cross-linked bone substrata or beads have been found to more efficiently capture viable cancer cells from a tissue fluid such as blood. Again, the bone substrata or beads are used as mimic of a natural matrix substrata that captures cancer cells and a small fraction of hematopoietic cells from blood or other tissue fluids related to metastasis, and can be used to detect those cancer cells and small fractions of hematopoietic cells.

In other embodiments, surfaces of the core materials are activated directly with bifunctional crosslinkers such as glutaraldehyde, washed and blocked with blood-borne adhesion components that promote the adhesion of cancer cells, such as, for example, 0.01-0.5 milligram per milliliter of human plasma fibronectin, fibrin, heparin, laminin, tenascin, vitronectin, or synthetic compounds, such as synthetic fibronectin and laminin peptides and the like in sterile and non-leaking conditions. The core materials including, but not limited to, bone, glass, inert polymeric materials, such as magnetic colloid, polystyrene, polyamide material like nylon, polyester materials, cellulose ethers and esters like cellulose acetate, urethane foam material, DEAE-dextran, as well as other natural and synthetic materials, such as other foam particles, cotton, wool, dacron, rayon, acrylates and the like. The blood-borne adhesion components-coated core materials are used as mimic of a natural matrix substrata that captures cancer cells and a small fraction of normal cells that are related with metastasis, and may be used to detect such cells.

In yet other embodiments, forms of denatured collagens, called gelatin, are used to coat core materials including, but not limited to, bone, glass, inert polymeric materials, such as magnetic colloid, polystyrene, polyamide material like nylon, polyester materials, cellulose ethers and esters like cellulose acetate, urethane foam material, DEAE-dextran, as well as other natural and synthetic materials, such as other foam particles, cotton, wool, dacron, rayon, acrylates and the like. The gelatin-coated core materials are then crosslinked with glutaraldehyde, washed and blocked with blood-borne adhesion components that promote the adhesion of cancer cells, such as, for example, 0.01-0.5 milligram per milliliter of human plasma fibronectin, fibrin, heparin, laminin, tenascin, vitronectin, or synthetic compounds, such as synthetic fibronectin and laminin peptides and the like in sterile and non-leaking conditions. Once more, the gelatin-coated core materials crosslinked with glutaraldehyde and blocked with blood-borne adhesion components are used as mimic of a natural matrix substrata that captures cancer cells and a small fraction of hematopoietic cells that are related with metastasis, and may be used to detect such cells.

Co-pending application PCT Patent Application PCT/US01/26735 (claiming priority to U.S. Provisional Patent Application No. 60/231,517) discloses that the cell-adhesion matrices may comprise core materials comprising collagenous fibers, fibrin gels, purified cotton or plastic fibers. The present invention discloses that many more core materials may be used. Some of these core materials have been found to be able to be coated without an intervening intermediate layer with purified human plasma fibronectin or its fragments.

The modified matrix may be contacted directly with the fluid from which the metastatic cancer cells are to be isolated, or may be applied as a thin coating to a cell separation vessel, such as a filter, tube, capillary, culture plate, cell isolation column, a flask etc., that are preferably sterilized. The thin coating is preferably immobilized to the cell separation vessel. The matrix-coated surfaces of the cell separation vessels are preferably designed maximize surface contact area. Beads, microbeads, or microcarriers may be used as a core material in order to increase the surface area available for contacting cells. The core material may also be in the form of micromeshes and/or packed beads. Matrix-coated beads and micromeshes form filtration channels to maximize contact areas between matrix and cells improving cell separation efficiency.

The modified matrix may be used to remove metastatic cancer cells and hematopoietic cells related to metastasis from a number of tissue fluids including, but not limited to, blood, bone marrow, ascites, lymph, urine, spinal and pleural fluids, sputum, airway and nipple aspirates. The cell separation method of this invention may also be used to isolate such cells from dissociated tumor tissue specimens and cultured tumor cells. Cancer cells that may be isolated using the modified matrix include, but are not limited to, carcinoma cells of prostate, breast, colon, brain, lung, head & neck, ovarian, bladder, renal & testis, melanoma, liver, pancreatic and other gastrointestinal cancer. Cancer cells that are particularly desired to be isolated include lung carcinoma cells, lung adenoma cells, colon adenocarcinoma cells, renal carcinoma cells, rectum adenocarcinoma cells, ileocecal adenocarcinoma cells, gastric adenocarcinoma, pancreatic carcinoma, hepatoma cells, hepatocellular carcinoma cells, prostate adenocarcinoma cells, bladder carcinoma cells, breast carcinoma, ovarian carcinoma, teratocarcinoma, amalanotic melanoma cells, malignant melanoma cells, squamous cell carcinoma of the cervix, esophagus, head & neck, air-way, larynx and of oral origin; glioblastoma cells, and endometrial adenocarcinoma cells. The present invention provides effective cell separation methods for diagnostic and therapeutic applications in patients with metastatic diseases, including, but not limited to, prostate, breast, colon, brain, lung, head & neck, ovarian, bladder, renal & testis, melanoma, liver, pancreatic and other gastrointestinal cancer.

Cell separation is performed by contacting the tissue fluid with the modified matrix surface. Tissue fluids such as whole blood, buffy coat, bone marrow, ascites, and lymph are treated with anticoagulants to prevent coagulation during the cell separation procedure. For examples, blood and buffy coat may be pre-diluted with one tenth volume of medium containing 0.5 mM EDTA or with anticoagulant citrate dextrose (ACID; Baxter Healthcare Corporation, IL) containing 50 unit heparin/ml.

The modified-matrix of the present invention can capture “viable” cancer and the small fraction of hematopoietic cells circulating in the blood involved in metastasis, but has little affinity for over 99.99% of blood cells. The invention is based on the adhesive and invasive functions of cancer cells and the small fraction of hematopoietic cells involved in metastasis with respect to the modified matrix. Cancer cells that are isolated may be subjected to in vitro assays, demonstrating that they are viable, invasive and metastatic. As the matrices of the present invention are non-toxic they can also accommodate the growth of isolated cells. The matrix facilitates cell separation enabling one to count the number of isolated viable cells, analyze genomic changes, profile gene expression and proteomics, and treat the tissue fluid where targeted cells are present in very low concentrations. The sensitivities can be on the order of 1 cell to 1 gram of sample.

It may be desired that the separated cells remain viable. For example, it may be desired to reuse certain of the separated cells therapeutically, or to grow them (e.g. the metastatic cancer cells) in an in vitro culture in order to amplify a signal for vaccine development. Conventional techniques such as the use of antibody-affinity microbeads typically subject the cells to a complicated and traumatizing course which not infrequently has an injurious effect on the cells. Considering the very low occurrence of the target cells, this phenomenon is particularly distressing.

This invention also provides an efficient method wherein viable cells captured on the modified matrix can be released readily from the modified matrix by the use of digestive enzymes, including, but not limited to, trypsin/EDTA solution (purchased from GIBCO), collagenases and hyaluronases. Cell adhesion molecules of the modified matrix, including fibronectin, laminin, and vitronectin etc, are sensitive to digestion. These enzymes will cleave binding between the cells and the modified matrix, and release viable cells from the matrix into suspension.

The cell separation method of the present invention may be used for cancer diagnostic purposes, e.g. early detection, monitoring therapeutic and surgical responses, and prognostication of cancer progression. The enriched separated cancer cells can be used, for example, to determine the metastatic potential of the patient's cancer. The sensitivity and accuracy of measuring the metastatic potential of a cancer may be further enhanced using additional assays known to those of skill in the art, such as determining the tissue origin of cancer cells, measuring the angiogenic capabilities of the cells, and determining the degree of reduction in leukocyte count or complement association.

Prognosis and therapeutic effectiveness may also be adjudged by assays that count numbers of viable and metastatic cells in the blood or other tissue fluids during and post therapeutic intervention(s). For example, the modified matrix may be contacted with a blood sample from a cancer patient and the isolated cancer and hematopoietic cells associated with metastasis subsequently detected and quantified using a combination of antibody labeling and microscopic imaging or flow cytometry. Selection of chemotherapeutic regimen may be optimized by determining those regimens that most effectively, without undue side effects, reduce the number of cancer cells and hematopoietic cells associated with metastasis in the blood sample as detected by the matrix. Optimization of selection of chemotherapeutic regimen may also be performed by subjecting the isolated cancer and hematopoietic cells to a battery of chemotherapeutic regimes ex vivo. Effective doses or drug combinations could then be administered to that same patient.

The cell separation system of the present invention may also be used to detect whether a new compound or agent has anti-cancer activity. For example, the number of viable cancer cells in whole blood can be determined before and after the administration of the compound or agent, with compounds or agents significantly reducing the number of viable cancer cells in the blood after administration being selected as potential anti-cancer candidates. Comparing the metastatic potential of the cancer cells throughout the treatment can follow the efficacy of the agent. Agents exhibiting efficacy are those, which are capable of decreasing number of circulating cancer cells, increasing number of viable associated leucocytes (host immunity), and suppressing cancer cell proliferation.

The modified matrix of the present invention may also be used as a “cancer cell trap” that allows for the high yield and efficient removal of viable cancer cells from the tissue fluids. The cell separation method of the invention may be employed in respect of the autotransfusion of blood salvaged during cancer surgery, therapeutic bone marrow transplantation, peripheral blood stem cell transplantation and leucopheresis, in which autologous transfusions are done, from which contaminating cancer cells have been removed.

The enriched cancer cells and their specific clusters of surface antigens isolated using the modified matrix may be used in fusions with dendritic cells for cancer vaccine development. For example, the cancer cells of different carcinoma cancers may be subjected to ex vivo culture and expansion, and the cells used in whole, or purified for specific membrane structures or for specific antigens, to interact with dendritic cells to develop an effective tumor vaccine.

As would be understood by one of skill in the art, the cell fraction enriched for cancer cells isolated using the disclosed matrices may also be used as a source of DNA, RNA and proteins in genomic, gene expression and proteomic profiling studies, for further discovery of genes, proteins and epitopes characteristic of the metastatic cell phenotype.

Further, the described matrices may be used to prevent full blown cancer from occurring by removing cells capable of metastasis from the circulation.

### BRIEF DESCRIPTION OF THE DRAWINGS

FIG. 1A depicts a front sectional view of an upright vacuum blood collection tube coated along its internal surface with a modified matrix film capable of segregating cells associated with metastasis that may be used in the diagnosis of metastatic cancer;

FIG. 1B depicts an enlarged front sectional view of a portion of the upright vacuum blood collection tube of FIG. 1A illustrating viable cancer and hematopoietic cells captured by the modified matrix film coated on the glass core material;

FIG. 2A depicts a front sectional view of an upright vacuum blood collection tube containing cell separation beads coated with a modified matrix film and further comprising a separator for capturing and filtering the separation beads when the tube is inverted;

FIG. 2B depicts an inverted front sectional view of the vacuum blood collection tube of FIG. 2A showing the cell separation beads trapped in the filter separator;

FIG. 3A depicts a front sectional view of an upright vacuum blood collection tube containing cell separation microbeads or nanoparticles coated with a modified matrix film and having an intermediate magnetic coating;

FIG. 3B depicts a front sectional view of the upright vacuum blood collection tube of FIG. 3A wherein a magnetic separator is applied to the tube to segregate the cell separation microbeads or nanoparticles from the supernatant;

FIG. 4A depicts a three-dimensional view of a cell separation filter containing within an inner confinement area cell separation beads coated with modified matrix which may be used in diagnostics, therapeutics or treatment according to the invention;

FIG. 4B is an expanded view of the portion of cell separation beads designated in FIG. 4A, depicting the anastomosic channels formed by the cell separation beads within the inner confinement area;

FIG. 5 is a schematic representation of a method of the present invention providing for the isolation of cancer cells from tissue samples using a combination of the function-affinity cell separation of the invention and immuno-affinity purification.

### DETAILED DESCRIPTION OF THE INVENTION

Discussion

The present invention provides an improved cell separation substratum for separation of metastatic cancer and a small fraction of normal cells associated with metastasis from a tissue fluid sample. The improved cell separation substratum comprises a supporting core material, comprising, but not limited to, bone or tissue fragments, magnetic colloid, plastic, glass and stainless steel, coated with an intermediate coating comprising material that has affinity, or efficiently binds to another material having the affinity, to bind blood-borne adhesion components that promote the adhesion of cancer cells, such as fibronectin, fibrin, heparin, laminin, tenascin, vitronectin, or their fragments, and that has the ability to effectively coat the core material used in the matrix. The improved cell separation substratum, or matrix, may be used to coat areas of objects which are intended to be in contact with the tissues from which the metastatic cancer cells and small fraction of normal hemopoietic cells that are to be isolated, such as a blood collection tube, plate, or flask, the surface of beads, or the inner lining of a capillary or filter, or may comprise the material of the object itself as the core material and another substance as the intermediate coating material. For example, a gelatin solution (2.5% gelatin w/v and 2.5% sucrose w/v in PBS) may be first coated on inner wall of a blood collection glass tube and the gelatin film fixed with 1% glutaraldehyde, followed by PBS washing and masking by human plasma fibronectin, 0.1 mg/ml, in sterile condition. The modified matrix in such case would comprise glass coated with gelatin masked with human plasma fibronectin.

By permitting isolation of viable cancer cells in high efficiency (i.e., allowing one to isolate the relatively small number of cancer cells typically seen in most tissue samples), the present invention achieves a highly desirable objective, namely providing a method for the prognostic evaluation of subjects with cancer and the identification of subjects exhibiting a predisposition to developing metastatic cancer.

The invention encompasses a method for determining the number of viable metastatic cells derived from a cancer subject comprising:
(a) adding a tissue fluid sample to a cell separation vessel, wherein the wall contacting the fluid is coated with a modified matrix film under conditions sufficient to specifically bind to cancer cells and a small fraction of hematopoietic cells associated with metastasis;
(b) washing the matrix films and removing unbound cells;
(c) treating the cell-bound matrix films with proteolytic enzymes; and
(d) eluting bound cells from the matrix films onto a solid support to provide an enriched cell sample comprising cancer and the small fraction of hematopoietic cells.

The enriched cell sample may be used for detecting and counting the number of viable cancer and a small fraction of hematopoietic cells using microscopic imaging or flow cytometry, wherein a detection of increasing number of viable cancer cells is an indicator of cancer cells with metastatic potential, and increasing number of hematopoietic cells is an indicator for host immunity.

The enriched cell sample may also be used for identifying an agent that inhibits metastasis of cancer cells by detecting and counting the number of viable cancer and hematopoietic cells treated with exogenous agents. A decrease in the number of cancer cells in the presence of the test agent, as compared to the number of cancer cells detected in the presence of a vehicle control, identifies a compound that inhibits metastases formation. On the other hand, an increase in the number of hematopoietic cells in the presence of the test agent, as compared to the number of hematopoietic cells detected in the presence of a vehicle control, identifies a compound that has immune activity against metastases formation.

Metastatic cancer cells may be identified by particular functional assays including:
(a) the intake of collagen or matrix fragments;
(b) the intake of acetylated low density lipoprotein (acLDL);
(c) the capacity of continued growth in culture in conditions containing complement-inactivated human sera; and
(d) the recognition by antibodies against both epithelial and endothelial markers but not by antibodies against leukocyte/monocyte common antigens such as CD14, CD45, and CD68.
   Enumeration of metastatic and hematopoietic cells in a given sample may be performed either by microscopic imaging or flow cytometry.

In accordance with one aspect of the invention, a crosslinked gelatin film may be prepared using the three following steps (a) to (c):
(a) gelatin is prepared and isolated from connective tissues of human or other animals;
(b) core material is covered with gelatin;
(c) the gelatin is crosslinked and the functional groups from the crosslinking agent are blocked with fibronectin.
   Gelatin may be crossed-linked as described in Chen and Singer, 1980; Chen et al., 1994. The gelatin-crosslinking method can be modified by persons of ordinary skill in the art to produce a gelatin-coating film having an affinity to viable cancer cells and a specific subset of hematopoietic cells associated with such viable cancer cells.

In one embodiment of the invention, there are provided cell separation beads comprising the modified-matrix. The core material of the beads may comprise, without limitation, bone, glass, inert polymeric materials, such as magnetic colloid, polystyrene, polyamide material like nylon, polyester materials, cellulose ethers and esters like cellulose acetate, urethane foam material, DEAE-dextran, as well as other natural and synthetic materials, such as other foam particles, cotton, wool, dacron, rayon, acrylates and the like. The beads preferably have a diameter in the range of 100 microns to 1,000 microns. The beads are coated on their surface to form a modified matrix having tremendous surface areas for contacting cells in the fluid. To enhance handling of beads in fluid, the core can have an intermediate magnetic coating, allowing the beads to be subsequently separated from tissue sample, and/or from each other, in a magnetic field. The cell separation beads can be placed into a blood collection tube, plate, flask, capillary, etc. for providing a confined area in which the beads may contact the cells in the fluid.

The cell separation beads are preferably 100 microns to 1,000 microns in diameter and may be coated with a crosslinked gelatin film. In one embodiment the crosslinked gelatin-coated beads are housed within a sterile vacuum blood collection tube with anticoagulant powder containing lithium heparin. In such embodiment approximately, 0.1-mL of gelatin-coated beads are used for every 5-ml blood that is to be collected. The blood-bead mixture in the tube is placed on a shaker set at slow speed at 37° C. for 30 minutes to 2 hours. The beads are then washed and collected using a mesh filter, preferably having mesh-opening widths of 75+/−12 microns.

The cell separation beads may be used to isolate cells associated with metastasis using the following method:
(a) adding a tissue fluid sample to a vessel containing the cell separation beads under conditions sufficiently allow the beads to bind to cancer cells;
(b) washing the beads and removing unbound cells through the use of a filter, such filter preferably having mesh opening widths of 75+/−12 microns;
(c) treating the cells-bound beads with proteolytic enzymes; and
(d) eluting bound cells from the beads onto a solid support to provide an enriched cell sample comprising cancer cells and typically a small fraction of hematopoietic cells associated with metastasis.

Alternatively the core material may comprise fibers. Fibers selected must be inert and compatible with the blood, and should be somewhat stiff to adhere well to the coating material, such as gelatin film. Preferably in a blood filter using fibers as its core material, the size of fibers should not typically exceed about 2 cm long, and should range from 10 microns to 1,000 microns in diameter. In blood filters, if the fibers are too big or too long, they can compact at high flow rates and less channel surface areas, and, therefore, be less efficient. In blood filters, the nature of fibers should be selected such that the fibers may adhere to the coating material and create a smooth anastomosic channel within the filter for blood flow. In forming a preferred filter, fibers may be packed tightly between two layers of meshes having mesh opening widths of 50 to 100 microns.

In magnetic cell separation applications involving the binding of microbeads or nanoparticles to cell surfaces, the microbeads or nanoparticles have a diameter in the range of 20 nm to 20 microns. The cell separation microbeads or nanoparticles may be directly coated with cell adhesion molecules using an attachment agent such as glutaraldehyde to activate binding of cell adhesion molecules to the surface of magnetic colloid microbeads or nanoparticles. In one embodiment the blood borne-cell adhesion molecules-coated microbeads are housed within a sterile vacuum blood collection tube with anticoagulant powder containing lithium heparin. In such embodiment, approximately 50 millions of the modified matrix coated microbeads or nanoparticles are used for every 5-ml blood that is to be collected. The blood-microbead mixture in the tube is placed on a shaker set at slow speed at 37° C. for 30 minutes to 2 hours. The microbeads are then washed and collected by passing the sample through a magnetic field to magnetically immobilize cells-microbeads mixture. The cell separation magnetic microbeads or nanoparticles may be used to isolate cells associated with metastasis using the following method:
(a) adding a tissue fluid sample to a vessel containing the cell separation magnetic microbeads or nanoparticles under conditions sufficiently allow the microbeads or nanoparticles to bind to cancer cells;
(b) washing the microbeads or nanoparticles and removing unbound cells by passing the sample through a magnetic field to magnetically immobilize microbeads or nanoparticles in the sample having cells bound thereto to provide an enriched cell sample comprising cancer cells and typically a small fraction of hematopoietic cells associated with metastasis.

A cell separation filter system comprising a pre-filter and the modified-matrix of the present invention may also be used to separate metastatic cells from a tissue fluid sample preferably presented as a fluid suspension. Such a cell separation filter system may be fabricated using the following steps:
(a) building a pre-filter and a connecting tube;
(b) packing a filter container with a filtration unit containing core materials comprising one or more of fibers, meshes and beads;
(c) bringing the pre-filter and filtration units into contact with a coating solution capable of coating the core material;
(d) removing the surplus amount of the coating solution;
(e) drying the coating solution on fibers, meshes and packed beads;
(f) crosslinking the coated film; and
(g) conjugating a cell adhesion molecule to the film surface.
   Such cell separation filter system therefore comprises a pre-filter, called the clump screen, preferably having a mesh pore width of 150 to 500 microns, a connecting blood tube and a filter housing. In a preferred embodiment, the filter housing comprises: an inlet in the housing for the introduction of the blood to be filtered; an outlet in the housing for the removal of filtered blood or to return to a patient; and a filter element disposed within the housing, which element comprises core materials of beads, preferably having a diameter in the range of 100 microns to 1,000 microns, and/or fibers ranging from 10 microns to 1,000 microns in diameters. Preferably the core beads or fibers are packed between two layers of meshes, having mesh-opening widths of 50 to 200 microns. The surfaces of both pre-filter screen and the filter element are coated with a material that has affinity, or efficiently binds to another material having the affinity, to bind blood-borne adhesion components that promote the adhesion of cancer cells, such as fibronectin, fibrin, heparin, laminin, tenascin, vitronectin, or their fragments, and that has the ability to effectively coat the core material, so that the pre-filter screen has affinity to cell clumps, called emboli, containing viable cancer cells, and the filter element contains anastomosic channels of tremendous surface areas for contacting cells in the fluid. The coated surfaces lining the anastomosic channels selectively remove viable cancer cells from the blood or other tissue fluids to be filtered.

The cell separation filter system may be used in a manner to remove cancer cells and emboli derived from blood or other tissue fluids of a cancer system using the following method:
(a) passing a tissue fluid sample through the cell filtration system, in which pores of the pre-filter and anastomosic channels of the filter comprise a modified matrix specific for adhesion and invasion by cancer cells and emboli but not by majority of tissue cells, wherein pores of the pre-filter and channels of the filter are under conditions sufficient to specifically bind cancer cells;
(b) allowing a substantial part of cancer cells and emboli to be entrapped in the cell filtration system; and
(c) removing adherent cancer cells and emboli; or returning the filtered blood to the patient as needed.

A preferred cell separation filter system contains a pre-filter, preferably having screen meshes with a pore width of 200 microns, positioned between a blood reservoir and the filter housing. The lining of pores in the pre-filter is coated with a crosslinked gelating film(s). The pre-filter removes large clumps form blood containing cancer cells (such large clumps can clog blood flow). The pre-filter unit may be disposable and can be modified form of the helically wound blood filter described in U.S. Pat. No. 4,092,246 (comprising sheet material having a pore width of 200 microns wound into a helical coil of desired tightness).

The cell separation filter system containing the pre-filter may also be used as a blood filter by subjects having metastatic cancer. The use of such a filter system involves the perfusion of the subject's blood through the modified-matrix anastomosic channels in the filter. In a preferred protocol, the subject's blood is withdrawn and are passed in contact with the modified matrix. During such passage, cancer cells present in the patient's blood preferentially adhere to the matrix and are removed from the circulation of a patient.

In a specific embodiment of the cell separation filter system useful for filtering metastatic cancer cells from a patient's blood, the pre-filter and the filter are formed within a containment vessel. The containment vessel is connected to a blood input line which is operatively coupled to a conventional peristaltic pump or to a gravity-dependent blood flow system. A blood output line is also included. Input and output lines are connected to appropriate arterial or venous fistulas, which are implanted into, for example, the forearm of a subject. Citrate-phosphate-dextrose anticoagulant is automatically added into the blood flow in an appropriate ratio. Alternatively, apheresed peripheral blood can be applied in conjunction with the cell filtration system. Apheresis is initiated upon recovery of the white blood cell count to equal or more than 1×10⁹/L. Apheresis or leucopheresis can be performed using a Cobe Spectra Cell Separator (Lakewood, Colo.) at a rate of 80 ml/min for 200 min (total volume of 16L).

A method of preventing metastases formation in a cancer subject using such blood filter comprises:
(a) inoculating a cancer cell sample derived from a cancer subject onto the cell filtration system;
(b) incubating the cancer cell sample for a time sufficient to allow adhesion of cancer cells to the coated pores of the pre-filter and anastomosic channels of the filter; and
(c) returning the filtered blood to the cancer patients.

Intraoperative autotransfusion of blood during major surgical procedures for removal of primary tumors and bone marrow transplantation for immunotherapy can be applied. The salvaged blood samples such as blood harvested from patients undergoing abdominal surgery for resection of primary cancers are passed through the cell filtration system of the present invention in conjunction with a commercial gravity-dependent blood device such as OR Bloodbanker autotransfusion system (International Technidyne, Edison, N.J.) or the Cell Saver (Haemonetics, Natick, Mass.). Citrate-phosphate-dextrose anticoagulant is automatically added into the salvaged blood in an appropriate ratio. The use of the cell filtration system of the invention provides a novel method that can remove viable and still invasive cancer cells from the salvaged blood and bone marrow, which provides potentially significant clinical benefit of autotransfusion and bone marrow transplantation to cancer patients.

The invention encompasses a method for isolating metastatic and angiogenic cells from a cancer subject comprising:
(a) passing a tissue fluid sample through the cell filtration system, wherein pores of the pre-filter and channels of the filter are under conditions sufficient to specifically bind cancer cells and emboli;
(b) washing the pre-filter and the filter and removing unbound cells;
(c) treating cell-bound anastomosic channels and pre-filter screen with proteolytic enzymes; and
(d) eluting bound cells and emboli from the pre-filter and the filter onto a solid support to provide an enriched cell sample comprising cancer cells and emboli.

Given the ability of such modified-matrix filters to isolate viable cells involved in metastasis and angiogenesis, the cells isolated by the present invention provide cellular sources for the discovery of cellular genes, RNAs, proteins and antigens important for prevention and intervention of metastases formation in a cancer subject.

For example, DNA microarray technology has been used advantageously in the identification of numerous genes differentially expressed in ovarian tumor samples (Welsh et al., 2001; Su et al., 2001; Giordano et al., 2001). From these studies, many genes have emerged as promising biomarker candidates, including HE4, a secreted protease inhibitor. Using a specialized array, many angiogenesis genes were found differentially regulated in ovarian cancer. In addition, serial analysis of gene expression (SAGE) was used to identify up-regulated genes in ovarian cancer, including Kop, SLPI, claudin-3 and claudin-4, making these products attractive candidate biomarkers. However, few have been linked to cancer progression and metastasis. A major problem encountered in linking the same as been the inability to obtain highly purified cancer cells to be used in the analysis. The fact is that tumors are composed of lots of different cell types. Many genes expressed at different levels are actually coming from non-tumor cells. A second major problem in linking up-regulated genes in ovarian cancer to cancer progression and metastasis is related to the viability of the cells. Apoptotic and necrotic tumor cells are common in larger tumor and ascites. A third major problem has been the lack of information concerning the invasive phenotype of cells under investigation. In order to understanding gene expression patterns of cells during cancer progression and metastasis, it is, thus, necessary to separate the viable from the dying cancer cells, the aggressive from benign cells, and the cancer cells from the normal cells in tumor samples. The present invention provides a method for separating and concentrating metastatic cancer cells.

It is known in ovarian cancer, that cancer cells can be found in primary organs, in ascitic fluid blood or lymph, and in peritoneal micrometastases. Cancer cells shed in ascites and blood are numerous and they can be obtained by non-invasive means. It is postulated that only a small fraction of cancer cells in ascites or blood may exhibit ability to adhere to and invade connective tissue barriers, and have potential for metastasizing to a new site. These rare cancer cells in ascites or blood are considered as “metastatic” cells, which when grown in collagenous matrix may mimic micrometastases and be considered as “metastasized” cells. By using the present invention to isolate metastatic cancer cells, a DNA microarray can be used to select robotically several sets of transcripts that were enriched in different purified viable cell types to address important questions of cancer progression and metastasis:
(i) higher in metastasized cells than in metastatic cells, indicating potential genes driving the process of extravasation;
(ii) higher in metastatic cells than in primary tumor cells, indicating potential genes driving the process of intravasation;
(iii) higher in both metastatic and tumor cells than in normal epithelial cells, suggesting genes encoding early markers for cancer progression; and
(iv) higher in both metastatic and tumor cells of ovarian epithelial cancer than in cancer cells of other diseases, i.e., endometrioma or colon adenocarcinoma, suggesting genes encoding possible cancer markers of tissue origin.
   The selected genes can be confirmed for their role in cancer progression and metastasis by a quantitative analysis using real time PCR on different cell types derived from normal, tumor and metastatic tissues. By a combination of DNA microarray and real time PCR, novel molecular markers and therapeutic targets for ovarian cancer can soon be discovered. Not only could the un-identified gene changes provide good targets for chemotherapeutic drugs, but they may also provide molecular markers to help clinicians assess tumor aggressiveness.

As would be understood by one of ordinary skill in the art, the present invention would likewise find use in other cancer types in characterizing the roles of genes, proteins, RNAs and antigens in cancer progression and metastasis.

### EXAMPLES

### Example 1

Preparation of Crosslinked Gelatin Films.

The following method may be followed to prepare crosslinked gelatin films useful in respect of preparing a modified matrix embodiment of the present invention:
(a) gelatin is isolated from connective tissues of human or other animals
   Type I collagen is purified from connective tissues of rat tails or human placenta and heat-denatured by boiling for 5 minutes. The gelatin solution is then allowed to dry at 100° C. in an oven under vacuum. Gelatin powders include these produced by acid- or heat-extraction and these from commercial sources including, but not limited to, heat-denatured bovine type I collagen type A derived from porcine skin, Sigma Chemical Co., St. Louis, Mo., USA.
(b) Core materials are coated with gelatin
   Gelatin powders are washed with chill distilled water three times by stirring and centrifugation of the gelatin particles. The gelatin solution, containing 2.5% gelatin w/v and 2.5% sucrose w/v, in PBS, pH 7.2, is heated until boiling for five minutes to completely dissolve gelatin particles. To coat a cell separation vessel, the gelatin solution is maintained at 45° C., overlays the core materials, and immediately removes excess gelatin fluid to leave a thin film covering the core materials. The gelatin film is left at 45° C. for 30 minutes until dried.
(c) The gelatin is crosslinked with a crosslinking agent and the functional groups on the crosslinked gelatin due to the crosslinking agent are blocked with fibronectin
   The gelatin film-coated vessel walls are placed in a chill 1% aqueous glutaraldehyde solution. The mixture is kept at ambient temperature for one to 24 hours and with weak agitation. The fixed films are washed several times with distilled water to eliminate the excess glutaraldehyde. The absence of reagent in the floating matter resulting from washing is checked by measuring the optical density at 280 nm (adsorption wavelength of glutaraldehyde).
   The free functions of the glutaraldehyde on the fixed gelatin film are then blocked with fibronectin. The films are incubated in PBS containing 0.1 mg human plasma fibronectin (Collaborative Research, Inc., Bedford, NIA). The solution is maintained at 20-37° C. for 20 minutes to 2 hours. To eliminate the free excess fibronectin present in the floating matter, the gelatin films are then washed (several times) with distilled water.

As would be understood by one of ordinary skill in the art given the present disclosure, other embodiments using core material coating-agents other than gelatin, such as, but not limited to, fibril collagens, fibrin and hyaluronates or synthetic polymers such as dextran and crosslinked fibronectin fragments, may be used without exceeding the scope or departing from the spirit of the invention. In addition, other cell adhesion molecules having fibronectin-like activities, such as laminin, fibrin, heparin and vitronectin (Collaborative Research, Inc., Bedford, Mass.) or their fragments, can be used as blocking agents for the crosslinked gelatin films. Accordingly, it is to be understood that this example disclosure is proffered to facilitate comprehension of the invention, and should not be construed to limit the scope thereof.

In accordance with one aspect of the invention, cancer cells and hemopoietic cells associated with metastasis may be separated and analyzed using the following steps:
(a) blood or buffy coat are prepared as sources of cells,
(b) viable cancer cells and a fraction of normal cells are separated on a cell separation vessel comprising the modified matrix film, and
(c) cancer and related normal cells are detected and total cells for each type counted.

### Example 2

Blood Cell Separation Using the Modified Matrix Film.

(a) Blood or buffy coat are prepared as sources of cells
   Five to ten ml of blood are drawn from control subjects or patients with a diagnosis of the presence of primary tumor or metastatic cancer into a blood collection tube (Vacutainer, Becton Dickinson, green top, each tube holds 7-ml) containing lithium heparin as an anticoagulant. Blood or cells collected from an in vivo source are subjected to cell isolation within a relatively short time after their collection because the cells may lose their viability. In order to maintain the optimal isolation of cancer cells, it is preferred that blood or tissue samples are stored at 4° C. and used within 24 hours after their collection, most preferably, within four hours.
   Buffy coat is processed from blood by conventional density gradient centrifugation using Ficoll-Paque (Pharmacia) that removes the majority of red cells leaving a thin layer of nucleate cells, called buffy coat, which may contain cancer cells of interest.
(b) Viable cancer cells are isolated on a cell separation vessel comprising the modified matrix
   The buffy coat is washed, and the nucleate cells are suspended in the complete cell culture medium, consisting of a 1:1 mixture of Dulbecco's modified Eagle's medium (DMEM) and RPM11640 supplemented with 10% calf serum, 15% Nu-serum (Collaborative Research, Inc., Bedford, Mass.), 2 mM L-glutamine, 0.1 mM non-essential amino acids, 1 mM sodium pyruvate, 1 unit/ml penicillin, and 10 ug/ml streptomycin. The cells are seeded onto a 6-cm tissue culture plate (NUNC) that were coated with the gelatin film. The cell culture is then incubated in CO₂ cell incubator for 30 minutes to 2 hours, and is washed gently with PBS to remove non-adherent cells.
   The adherent cells on the matrix film are then suspended with trypsin/EDTA solution (GIBCO) for 5 minutes, followed by washing with complete medium. Cells in the washes are the enriched cell sample comprising cancer and a small fraction of hematopoietic cells that are frequently related to metastasis.
(c) Detection of cancer cells and total cell count
   The enriched cell sample is used for detecting and counting the number of viable cancer and small fraction of hematopoietic cells related to metastasis using microscopic imaging or flow cytometry.

Detection of increasing number of viable cancer cells is an indicator of cancer cells with metastatic potential, and increasing number of hematopoietic cells isolated by the matrix is an indicator for host immunity. The metastatic cancer cells may be identified by functional assays described below.

### Example 3

Identification of Viable Cancer Cells

(a) Colony Formation

A portion of enriched nucleate cells, i.e., equivalent to 0.1-ml blood volume per well, are seeded onto a 16-well microtiter plate-glass slide (in 96-well microtiter plate format; Lab-Tek, Rochester, N.Y.) comprising tissue culture medium containing 10% heat-inactivated human plasma (complement-inactivated human sera) or plasma. Cells are allowed to propagate for four days to two weeks thereby allowing the cancer cells to form colonies. It was estimated that, among approximately 100 putative metastatic cells isolated from the blood of patients with metastatic diseases, there was only one colony of carcinoma cells formed after one week of culture. The efficiency of colony growth in culture appears to be 10,000 folds higher than what was observed in vivo, suggesting that, free of host immunity, cultured cancer cells increase their capability to grow.

(b) Apoptosis and Cytolysis

Cells are cultured for one day and stained prior to fixation using Vybrant Apoptosis Assay Kit #5 Hoechst/prodidium iodide (V13244, Molecular Probes, OR, USA). Within one day after isolation using the cell separation technology of the invention and in culture, approximately 1,000 putative metastatic cells and 100,000 leukocytes are isolated from one milliliter whole blood (containing approximately 10,000,000 nucleate white cells and 1,000,000,000 red cells) of patients with metastatic diseases. Viable cancer cells are resistant to Hoechst staining of nucleic acids within the cells, and do not uptake prodidium iodide while apoptotic or lysed cancer cells are stained with Hoechst staining. All leukocytes become apoptotic, as indicated by strong nuclear Hoechst staining, and some cells disintegrate, as indicated by red fluorescent prodidium iodide in the cells.

### Example 4

Identification of Metastatic Cancer Cells

(a) Intake of Collagen and Acetylated Low Density Lipoprotein

The intake of collagen or matrix fragments and that of acetylated low density lipoprotein (acLDL) by circulating cancer cells is indicative that the cells are invasive, angiogenic and metastatic.

Enriched cells are seeded on rhodamine-labeled collagen coated on a 16-well glass slide (Lab-Tek, Rochester, N.Y.). The cells are grown on the labeled collagen for 12 to 24 hours. The cells are then incubated with fluorescein-conjugated acLDL for 1 hour. The cells are then stained by nuclear staining with Hoechst dye for 10 minutes. For measurement of the invasive phenotype of these cells, cells were analyzed for the ability of the cell to adhere to, degrade and ingest rhodamine-collagen substratum. Metastatic cells exhibit extensive collagen-degrading/ingestion activities. Metastatic cells also exhibit the intake of fluoresceinacLDL, suggesting their role in angiogenesis, a process of metastasis. Neither leukocytes nor monocytes and endothelial cells exhibit these properties. Furthermore, immuno- and morphological features of metastatic cells are characteristic of carcinoma cells (see below).

(b) Immunocytochemistry

For the determination of possible developmental lineages of cancer cells, the enriched cells from blood of cancer patients are analyzed for their potential epithelial origin by immunocytochemistry using antibodies against epithelial specific antigen (ESA), epithelial membrane antigen (EMA; Muc-1), and cytokeratins 4, 5, 6, 8, 10, 13, and 18 (PCK). Commercial sources of antibodies for epithelial markers include mouse mAb recognizing human epithelial specific antigen (ESA; clone VU-1 D9, NeoMarkers, Calif., USA; SIGMA, MS, USA), Muc-1 epithelial membrane glycoprotein (Muc-1; clone E29, NeoMarkers, Calif., USA), cytokeratins 4, 5, 6, 8, 10, 13, and 18 (PCK; clone C-11, SIGMA, MS, USA). Furthermore, immunocytochemical staining using antibodies against endothelial markers, including CD31/PECAM-1 endothelial cell marker (CD31; Clone JC/70A, NeoMarkers, Calif., USA), Flk-1, a receptor for vascular endothelial growth factor (Flk-1, Clone sc-6251, Santa Cruz, USA), VE-cadherin endothelial marker (VE-cad; Clone sc 9989, Santa Cruz, USA); CD34 peripheral blood stem cell marker (CD34; clone 581, Pharmingen, USA), may be used to confirm the above observation that metastatic cells may process endothelial function. A preferred antibody staining is to use fluorescein conjugated antibodies against Muc-1 epithelial marker (EMA, DAKO, Denmark) or fluorescein conjugates of goat antibodies against factor VIII endothelial marker (F8; Atlantic), in the above functional labeling of cancer cells with rhodamine-collagen fragments to demonstrating the presence of both fluorescein-epithelial or endothelial markers (green fluorescence) and ingested rhodamine-collagen fragments (red fluorescence) in same cancer cells.

It was estimated that less than 1% of leukocytes and peripheral blood monocytes derived from cancer patients are co-isolated by the cell separation method of this invention. These hematopoietic cells are determined by antibodies directed against CD14, CD68 and CD45 leukocyte common antigen (CD45; clone T29/33, DAKO, Denmark).

In addition to the use of fluorescent labelings described above, alkaline-phosphatase-anti-alkaline-phosphatase (APAAP) method may be used to generate signals for antibody labeling. This allows one to visualize the cancer cells with their markers, and the cell morphology, by a high-resolution interference differential contrast (DIC) microscopy.

In one preferred embodiment of the present invention enriched cells are seeded on 16-well chambered glass slides (Lab-Tek, Rochester, N.Y.) coated with rhodamine-collagen. The seeded cells are cultured on the same substratum for 12-24 hours in a CO₂ incubator at 37° C. Prior to fixation for immunocytochemistry, the cells are incubated with fluorescein-conjugated acLDL for 1 hour, followed by nuclear staining with Hoechst dye for 10 minutes. After fixation with 3% paraformaldehyde in PBS, pH 7.2, for 10 minutes and after blocking nonspecific binding sites with 2% BSA for 30 minutes, mouse primary antibodies, or fluorescein-F8 or -Muc-1 (when fluorescein-conjugated acLDL is not involved) are applied to the slides. The samples are incubated for 20 minutes at room temperature, washed twice in PBS for 5 min, and then exposed to secondary rabbit anti-mouse Ig (Z0259, Dako) for another 20 minutes. After two more washes, the samples are incubated with alkaline-phosphatase-anti-alkalinephosphatase (APAAP) mouse Ig complexes for 15 min. Finally, the enzyme-substrate [NewFuchsin (Dako)] is added, resulting in the development of red precipitates at the cells of interest.

Data from the APAAP test may be recorded by numerous methods known to those of ordinary skill in the art, including by way of a Nikon Eclipse E300 inverted light microscope, or automatically scanning prepared slides using a Rare Event Imaging System (Georgia Instruments, Inc. (Roswell, Ga.)), in conjunction with a SONY DC5000 Cat Eye Imaging system. Data may be stored on a computer server or other device for later analysis. The Rare Event Imaging System employs image processing algorithms to detect rare fluorescent events and determine the total number of cells analyzed. It is comprised of an advanced computer-controlled microscope (Nikon Microphot-FXA, Nikon, Japan) with autofocus, motorized X-, Y-, and Z-axis control, motorized filter selection, and electronic shuttering. Images are taken by an integrating, cooled CCD detector and processed in a computer imaging workstation.

Most metastatic cells in an enriched cell sample react positively with ESA, Muc-1 or PCK and typically are of epithelial origin. Metastatic cells generally do not react with markers for leucocytes or monocytes, are usually larger than hematopoietic cells, and typically assume a carcinoma cell morphology on collagenous matrices. Circulating carcinoma cells are rare in blood of most normal donors, patients with benign disease, and cancer patients undergoing conventional chemotherapy. In the blood of cancer patients who are undergoing chemotherapy, both circulating cancer cells and leukocytes are generally not reactive to the modified matrix of this invention.

(c) Analysis by Flow Cytometry

In order to better enumerate single metastatic cells in the blood, an enriched cell sample of the present invention can be analyzed by flow cytometry following a manufacture's procedure. Alternately, cell samples containing individual cancer cells and emboli (clumps) can be automatically measured using a micro capillary fluorescent measurement system that can detect signals of both single cells and clumps.

Enriched cell samples may be stained for fluorescence sorting using procedures similar to these used in immunocytochemistry described above. The cells are determined for the metastatic propensity and apoptosis or cytolysis by cellular labeling prior to fixation using rhodamine-collagen substratum, fluorescein-acLDL and Vybrant Apoptosis Assay Kit #5 Hoechst/prodidium iodide (V-13244, Molecular Probes, OR, USA). In addition, the enriched cells are stained for cell type markers in a solution containing fluorescein-antibodies against Muc-1 (DAKO), fluorescein-antibodies against factor VIII endothelial marker (F8; Atlantic), phycoerythrin (PE)-conjugated anti-CD31 endothelial marker (Becton-Dickinson) and peridinin chlorophyll protein (PerCP)-labeled anti-CD45 (Becton-Dickinson) for 15 minutes. In general, three fluorescent labelings are applied to a given sample: including the rhodamine-collagen, a fluorescein-, PE-, or PerCP-labeled cell type signal, and a Hoechst staining. Briefly, the staining procedure involves incubation with fluorescent dye-antibody conjugates and washing. The labeled cells are re-suspended in 0.5 ml of a buffer and the sample is analyzed on a FACScan or FACSVantage flow cytometer (Becton Dickinson).

### Example 5

Determination of Efficiency of Recovery of Cancer Cells Using the Modified-Matrix

The human amelanotic melanoma cell line LOX was obtained from Professor Oystein Fodstad, Institute for Cancer Research, the Norwegian Radium Hospital, Oslo, Norway, and the human breast carcinoma cell lines MDA-MB-436 and Hs578T were obtained from American Type Culture Collection (Rockville, Md.). Cells were cultured in a 1:1 mixture of Dulbecco's modified Eagle's medium (DMEM) and RPMI 1640 supplemented with 10% calf serum, 5% Nu-serum (Collaborative Research, Inc., Bedford, Mass.), 2 mM L-glutamine, 0.1 mM non-essential amino acids, 1 mM sodium pyruvate, 1 unit/ml penicillin, and 10 ug/ml streptomycin.

LOX human malignant melanoma cells are tagged with a fluorescent dye, PKH26 Red Fluorescent Cell Linker (Sigma), to determine the efficiency of recovery of cancer cells using the cell separation procedure of this invention. Fluorescent-tagged LOX cells were cultured on fibronectin-coated crosslinked gelatin films for one day, suspended and counted the fluorescently labeled cells using a hemocytometer. They were serially diluted and spiked into complete medium alone, and in parallel into the blood of a control normal donor. Graded doses of LOX cells were seeded into 1 mL volumes of whole blood and complete medium, respectively, that were in 12-well culture plates that were coated with crosslinked gelatin films, and incubated for two hours. After washing with complete medium and PBS, the adherent cells were suspended by trypsin/EDTA (GIBCO). The enriched cell samples were further seeded onto a 16-well glass slide (Lab-Tek, Rochester, N.Y.), cultured for over three hours, and counted by fluorescence microscopy for the number of fluorescent LOX cells in each well. Samples were analyzed for the number of cancer cells per well and related to the total cell count per milliliter of blood.

The efficiency of recovery of fluorescent-tagged LOX cells from whole blood using the modified-matrix described above and cell separation procedure of this invention is shown in Table 1 below. Viable cancer cells were detected in blood samples, which initially contained as few as one cancer cell/mL (in three trials of the one cell experiment, two had detected one cell in the well). The result suggests that the level of sensitivity by the cell separation method is at 1 viable cancer cell per mL of blood. The recovery of viable cancer cells spiked into 1 mL of blood (10-20 million nucleate white cells and one billion red cells) from a normal donor, as compared with complete medium was consistent over a frequency range, from 63.3% to 89.9% at all cancer cell doses, and has an average recovery of 75.9%. It appears that high cell density in whole blood does not significantly affect the efficiency of the procedure. The average recovery rate (75.9%) can be used to estimate the number of viable cancer cells in the circulation.

TABLE 1
Efficiency of recovery of LOX cells from whole blood using the
cell separation procedure

| LOX cells/ml blood | LOX cells/ml medium | % Cells recovered |
|---|---|---|
| 8,545 | 9,834 | 86.9 |
| 2,193 | 2,440 | 89.9 |
| 1,054 | 1,213 | 86.9 |
| 547 | 612 | 89.4 |
| 248 | 299 | 82.9 |
| 61 | 83 | 73.5 |
| 28 | 44 | 63.6 |
| 13 | 20 | 65.0 |
| 7 | 11 | 63.6 |
| 4 | 6 | 66.7 |
| 2 | 3 | 66.7 |
| 0 | 0 | — |
| | | Average = 75.9 |

### Example 6

Use of Isolated Ovarian Cancer Cells for Discovery of Molecular Markers and Therapeutic Targets for Ovarian Cancer

(a) Purification of Ovarian Metastatic Cancer Cells

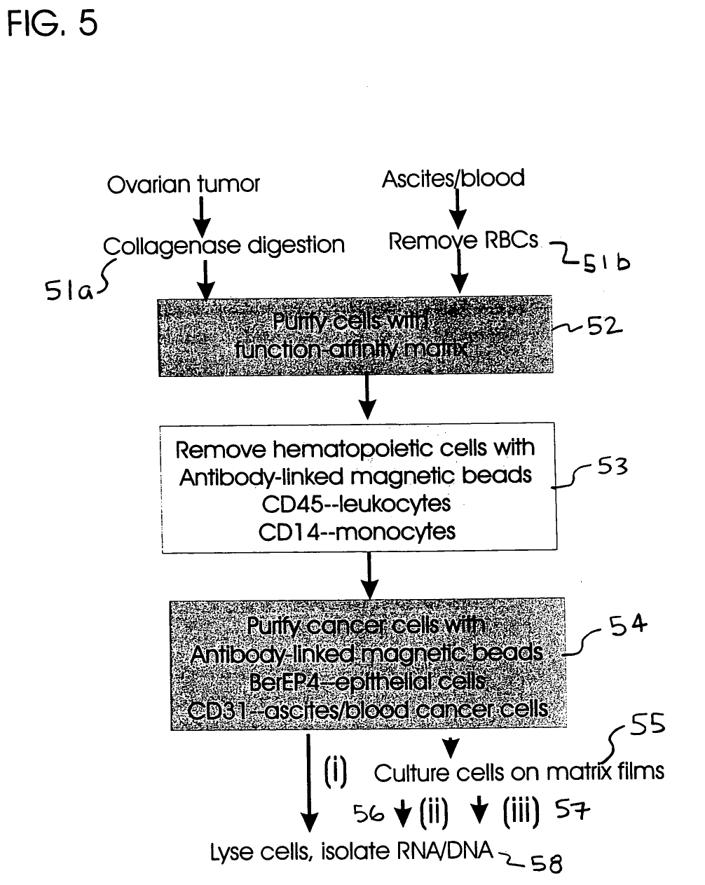

Ovarian cancer cells may be purified using a combination of function-affinity cell separation of the invention and immuno-affinity purification. FIG. 5 shows the scheme of such cell separation from bodily tissues, such as tumors, ascitic fluid or blood. Tumor and adjacent normal tissues optimally should be obtained immediately after surgical removal and digested with collagenase for 1 hour at 37° C. to yield a suspension of single cells and clumps (Step 51a). Ascites or salvaged blood samples, such as blood harvested from patients undergoing abdominal surgery for resection of primary cancers, preferably are removed of red blood cells by density gradient centrifugation procedure (Step 51b).

The first positive selection for purifying viable cancer cells involves passing tumor and ascites cell suspensions through the function-affinity matrix (Step 52). Briefly, cell suspension samples may be passed through a cell filtration system wherein pores of the pre-filter and channels of the filter contain materials that under specific conditions are sufficient to specifically bind cancer cells and emboli. The pre-filter and the filter are washed with PBS to remove unbound cells. The cells and emboli bound to the pre-filter and the filter may be released from the matrix by treating filter channels with proteolytic enzymes such as trypsin/EDTA, and the enriched cell sample collected. The enriched cells and other purified cells may be quantified, for example, using a hemocytometer.

The enriched cell samples may be further enriched by subjecting the sample to a negative selection procedure. For example, a cocktail of anti-CD14 and anti-CD45 immuno-magnetic beads (Dynal) may be used to remove hematopoietic cells as well as cancer cells binding non-specifically to the magnetic beads (Step 53).

The further enriched cell samples preferably are then subjected to a second positive selection procedure involving antibody-affinity purification. For example, the epithelial cells remaining in the cell suspensions may be isolated by binding to anti-BerEP4 immunomagnetic beads (Dynal) (Step 54), the BerEP4 antibody recognizing a pan-epithelial antigen present on normal and neoplastic epithelium but not present on hematopoietic or stromal cells (U. Latza, G. Niedobitek, R. Schwarting, H. Nekarda, H. Stein, 1990. J. Clin. Pathol. 43, 213). Importantly, the BerEP4 bound epithelial cells in ascites and blood express endothelial markers including factor VIII, CD31, and receptor for acetyl LDL, but BerEP4 bound primary tumor cells do not. Thus, the cancer cells in ascites and blood are also isolated by their binding to anti-CD31 immuno-magnetic beads.

Isolated cells may be lyzed and RNA/DNA isolated for further analysis. A portion of “metastatic” cancer cells isolated, as for example, from ascites and blood may also be cultured in a collagenous matrix (Step 55) for less than two days to give rise to a “metastasized” cell population mimicking cancer cells grown in micrometastases. Other steps (Steps 56 and 57), as would be known to those of ordinary skill in the art, could be performed to further improve the purity of the metastasized cell population.

In genetic studies, short-term cultures of ovarian surface epithelial cells may be used as the control normal epithelial cell group. Ovarian cancer-derived cell lines, SK-OV-3 [American Type Culture Collection (ATCC) HTB-77], MDAH-2774 (ATCC CRL-10303), and CAOV-3 (ATCC HTB-75), may be obtained from the ATCC and grown in DMEM (Life Technologies, Rockville, Md.), supplemented with 10% (vol/vol) FCS and penicillin/streptomycin. In addition, levels of gene expression of the above three cancer-cell types may be compared with those of stroma (fibroblastic) cells or leukocytes and monocytes to rule out potential normal cell-contamination in the cancer-cell preparation. The results of such comparison may be used to help discern patterns of gene expression that are consistent with cancer progression and development of the metastatic phenotype.

(b) Microarray Hybridization

Total RNA from the ovarian cancer cells isolated may be prepared with a Qiagen RNeasy mini-kit according to the manufacturer's instructions (Step 58). RNA may be hybridized separately to large microarrays containing 16,000 human genes (Affymetrix; U95A). Arrays may be scanned using an Affymetrix confocal scanner and analyzed initially, for example, using GeneChip 3.1 (Affymetrix) as set forth below.

(c) Microarray Data Analysis

Microarray scanned image files may be visually inspected for artifacts and analyzed with GeneChip 3.1 (Affymetrix) and GeneSpring 4.0 software (Silicon Genetics). Each image may be scaled to an average hybridization intensity of 200, which corresponds to approximately 3-5 transcripts per cell. The expression level (average difference) for each gene may be determined by calculating the average of differences in intensity (perfect match-mismatch) between its probe pairs. Genes with average hybridization intensities <0 across all samples may be excluded from further analysis. GeneSpring 4.0 software (Silicon Genetics) is used to select, group, and visualize genes whose expression varied across the samples with SD≧250. Hierarchical clustering of the samples and gene expression levels within the samples may be used to lead to the unambiguous separation of normal, primary tumor and malignant cells, as well as the identification of three subsets of ovarian cancer cell samples, i.e., primary, metastatic and metastasized.

To identify potential tumor markers, the hybridization intensity of each gene in normal and malignant cell samples may be compared, and three different estimates for population differences (difference of means, fold change, and unpaired t test) may be applied in parallel. The genes are ranked according to each metric, and the sum of the metrics was used to derive a semiquantitative estimate of the differential abundance of each transcript. Four categories of potential genes encoding molecular markers are:
(i) For a gene to be selected as enhanced during extravasation, it has to be expressed in all “metastasized” cell samples (BerEP4+ or CD31+ cancer cells from ascites or blood samples with culture) at least 5 times higher than in all “metastatic” cell samples (BerEP4+ or CD31+ cancer cells from ascites or blood samples without culture), with experiments done in duplicate.
(ii) For a gene to be selected as enhanced during intravasation, it has to be expressed in all “metastatic” and “metastasized” cell samples (BerEP4+ or CD31+ cancer cells from ascites or blood samples with and without culture, respectively) at least 5 times higher than in the tumor cell samples, with experiments done in duplicate.
(iii)For a gene to be selected as enhanced during ovarian cancer progression, it has to be expressed in all tumor, “metastatic” and “metastasized” cell samples at least 5 times higher than in the normal epithelial and stoma culture samples, with experiments done in duplicate.
(iv)For a gene to be selected as enhanced and as a specific marker for ovarian cancer, it has to be among genes fit in category (iii) above, and expressed at least 5 times higher than in all ascites “metastatic” and “metastasized” cell samples of endometrioma or other cancer, with experiments done in duplicate.

(d) Validation of Cell-Specific Gene Expression

Microarray results of differential expression of genes may be validated in at least three distinct ways. First, fragments of genes of interest may be amplified by RT-PCR from the RNAs of distinct cell types in triplicates to determine the overexpression of specific genes in specific cell types. Second, the National Center for Biotechnology Information (NCBI) “gene-to-tag” databases, available through UniGene (http://www.ncbi.nim.nih.gov/UniGene/), for gene expression patterns of these same three genes in tumor cells and tissues may be queried. LU and HE4 are typically highly expressed in primary ovarian tumors, as well as in other tumors and micrometastases. Third, quantitative large-scale analysis of gene expression in different cancer cell types may be performed using real-time RT-PCR as described below.

(e) Real-Time RT-PCR

To validate and extend previous findings of genes differentially expressed in ovarian tumor tissues, real-time RT-PCR, a highly sensitive and reproducible technique, may be chosen, preferably using robotic means, in validation of a potential set of markers for diagnostic and prognostic applications for treating patients with ovarian cancer (Hough et al., 2001). This method allows highly quantitative analysis of gene expression on a large number of specimens. In addition, it requires a relatively low amount of RNA, typically less than 1 pg. Real-time RT-PCR does not require large amounts of starting RNA in each purified cell type, and it can measure levels of gene expression of 32 RNA samples at one time. This approach would allow an accurate determination of the frequency and extent of overexpression of many genes relevant to ovarian cancer. The approach may also take advantage of genes selected from the vast screen assay of DNA microarray. Such genes may be tested stringently to determine those genes that are consistently and highly upregulated in a set of over 100 well-defined cancer cells from ovarian cancer in order to determine the “ovarian cancer gene cassettes” that are useful in diagnostic and prognostic applications for treating patients with ovarian cancer. Furthermore, real-time RT-PCR is feasible to be used in measuring the genuine up-regulated ovarian cancer genes in 5 mL blood of any patients who are in high risk of developing cancer.

In a typical procedure, one picogram of total RNA from each sample is used to generate cDNA using the Taqman reverse transcription reagents (PE Applied Biosystems, Foster City, Calif.). Mock template preparations are prepared in parallel without the addition of reverse transcriptase. Quantitative PCR is performed with an iCycler (Bio-Rad, Hercules, Calif.) using Pico Green dye (Molecular Probes, Eugene, Oreg.), and threshold cycle numbers are obtained using icycler software v2.3. Representative conditions for amplification are: one cycle of 95° C., 2 min followed by 35 cycles of 95° C., 15 sec, 58° C., 15 sec, and 72° C., 15 sec. Quantitative PCR reactions are typically performed in triplicate and threshold cycle numbers averaged. RT-PCR products should meet two criteria to be included in a study: (1) the signal from the reverse transcriptase (RT)-derived cDNA should be at least 100 fold greater than that of control reactions performed without reverse transcriptase, and (2) the PCR products from the reactions with RT should be the expected size upon gel electrophoresis. Gene expression may be normalized to that of beta-actin, a gene that is uniformly expressed in all ovarian cells as assessed by DNA microarray.

(f) Methods for Preparation and Analysis of DNA

Genomic DNA may be prepared from purified epithelial cells using the Qiagen DNA Easy Purification Kit (Qiagen). Preferably at least six independent replicates on each DNA sample are performed in order to assess gene copy number. Real-time PCR may be carried out as described above for the expression analysis, except that the control reactions should be carried out without any genomic DNA template. Appropriate primers may be used to design primers for genomic PCR, such as Primer 3 (http://www.genome.wi.mit.edu/cqi-bin/primer/primer3www.cqi). Representative conditions for amplification are: one cycle of 95° C., 2 min followed by 35 cycles of 95° C., 15 sec, 58° C., 15 sec, 72° C., 15 sec.

### Example 7

Functional Proteomics Studies to Aid in Diagnosis of Metastatic Phenotype and in Monitoring Chemotherapy Effect

Genomic methodologies described in EXAMPLE 6 provide significant information about gene structure and expression as well as other events such as splicing. However, the vast array of posttranslational modifications and surface localization commonly observed in proteins cannot be studied or be predicted accurately. Proteomic techniques are a solution to definitively study posttranslational modifications of abundant proteins but they alone have restricted value in understanding surface localization and interaction of minor functional proteins such as enzymes (Mann et al., 2001). To enrich minor proteins of specific function, advanced separation methodologies for isolating specific cell types as described above, membrane structures or protein complexes must be used in combination with sophisticated proteomic technology (Bell et al., 2001; Mann et al., 2001; Pawson and Scott, 1997).

2-D DIGE (Differential In Gel Electrophoresis)-mass spectrometry system (Amersham Pharmacia Biotech) may be used in conjunction with the function-based, cell separation method of the invention to facilitate the studies on molecular structures underlying the metastatic phenotype. To identify structure of membranes, an invadopodia membrane separation method (Mueller et al., 1999) may be used to isolate invadopodia proteins. To further enrich protein complexes of interest, affinity-based purification can be performed using immobilized antibody against the epitope, followed by competitive elution with peptide encoding the epitope as described, for example, in Mann et al., 2001. By proteomic analysis on a defined cell product exhibiting the metastatic phenotype, the targeted proteins and their endogenous inhibitors could be identified.

(a) Determination of Whether Natural Substrates and Inhibitors Associated with Seprase Exist as Enzyme-Substrate Complexes at Invadopodia

The 2-D DIGE-mass spectrometry system (Amersham Pharmacia Biotech) may be used for the identification of: (a) natural substrates (inhibitors) of a cell surface enzyme involved in cell invasion (a member of invadopodia proteases), called seprase, and (b) novel proteins associated with the enzyme in physiological complexes. Recent data from membrane purification and immunoprecipitation experiments suggest the existence of invadopodia complexes that contain seprase and form the structural basis for expression of the metastatic phenotype. However, there are many proteins involved, including proteases, their substrates in degradative process, integrins, kinases, cytoskeletal and signal molecules in their isoforms.

The 2D profiles of seprase-associated proteins in the presence of seprase inhibitors (experimental) and the absence of protease inhibitors (control) may be compared and analysed. For example, approximately 10⁹ LOX human malignant melanoma cells that express seprase and other invadopodia antigens are lysed in 0.15 M NaCl, 4% CHAPS, 30 mM Tris, pH 8.5. Proteins associated with seprase are immunoprecipitated using monoclonal antibodies directly conjugated on Agarose beads. A pair of immunoprecipitates are incubated at 37° C. in the presence (experimental) of and the absence (control) of seprase enzymatic inhibitors (5.0 mM ABESF, and 300 pM H-Ile-Pro-NHO-pNB). Proteins are then eluted from the column with 6 M urea, 4% CHAPS, 30 mM Tris, pH 8.5 (4° C.) that contain the epitope peptide, and concentrated by ultra filtration with MW 5,000 cut-off to a total protein concentration of approximately 0.5 mg/ml. These two samples are the control and experimental groups, and are labeled with 2 different Cy™ dyes developed for DIGE that do not have apparent alteration on electrical mobility of proteins, and resolved by 2D gel electrophoresis followed by mass spectrometric identification. Among approximately 400 analytic spots, approximately 50 spots are showing more than a 2-fold increase, and approximately 50 spots showing more than a 2-fold decrease are picked and their peptide sequences analyzed using MALDI MS to indicate the identity of putative natural substrates for seprase. Similarly, form those spots (among approximately the 400 analytic spots) that show less than a 2-fold change and that show sharp spot match, approximately 50 best-fitted spots may be picked and their peptide sequences analyzed using MALDI MS to indicate the identity of putative associated proteins for seprase that are involved in cell invasion.

The 2-D DIGE system is high throughput and ideal for complex analysis. In a preliminary study described above, immuno-affinity purified proteins derived from malignant human melanoma cells (LOX cell line) detect 428 analytical spots in a 2D gel, suggesting the feasibility of using the 2D DIGE system in such a complex analysis. Interestingly, tenascin-X was identified as a potential natural substrate for seprase as it has 5-fold higher amount in the seprase-complex treated with seprase inhibitors.

(b) The Monitoring of Ovarian Cancer Therapy Using Functional Proteomic Studies

In order to determine the efficacy of ovarian cancer therapy, the 2D profiles of cell-matrix contact membranes (including invadopodia that invade in the collagenous film) derived from metastatic cells described above, in the presence of therapeutic agents ex vivo (experimental) and in the absence of therapeutic agents ex vivo (control), may be compared and analyzed. For example, the experimental group may comprise cells cultured ex vivo in the presence of conventional Taxol/carboplatinum chemotherapy (Taxol 175 mg/m2 over 3 hours, carboplatinum AUC=7.5) or experimental therapeutics such as angiogenesis-MMPI (AG3340 Agouron/Warner-Lambert; Bay 12-9566 Bayer; or Marimastat British Biotech), while the control group may comprise cells cultured in the absence of therapeutic agent.

A 2-D DICE and mass spectrometric analysis may be performed on proteins from both experimental and control groups. Briefly, cell membranes (invadopodial membranes) in contact with a collagenous matrix are isolated and membrane proteins are partitioned into Triton X-114 according, for example, to the method described in Mueller et al., 1999. This procedure can be used to produce invadopodial membranes having 51% purity as determined by morphometry and immuno-labeling, and 122-fold enrichment over the membranes for the invasiveness marker seprase. The control and experimental membrane proteins are cyedyed with 2 different dyes and run upon a single 2D gel. Approximately 50 spots from those that show the highest increase, and approximately 50 spots showing highest decrease in the comparison, are picked and their peptide sequences analyzed using MALDI MS to indicate the identity of proteins associated with expression of the malignant phenotype. Resulting major membrane proteins are used to assess the overall proteomic profiling and to correspond to known invadopodia residents, proteases (seprase and MT1-MMP), and integrins, α3β1 and α5β1 for cell surface proteolytic cascades and integrin signaling pathway, respectively. The goal is to resolve functional proteomics of cancer malignancy by correlating the identification and analysis of invadopodia proteins to the function of genes or proteins. This approach may be used to provide information that may help develop targeted therapeutic agents.

### DESCRIPTION OF THE FIGURES

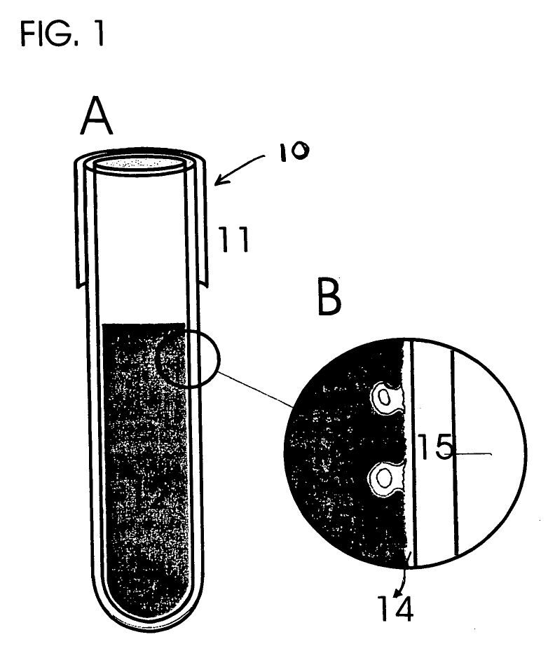

Now turning to the figures, referring to FIGS. 1A and 1B there is shown a general schematic of a contained cell separation system (10) comprising a vacuum blood collection tube (11) in which whole blood (12) may be stored. Vacuum blood collection tube (11) is coated along its inner surface of the tube's walls (15) with a modified-matrix (14) comprising core material, such as inert glass and polymeric materials, such as magnetic colloid, polystyrene, polyamide material like nylon, polyester materials, cellulose ethers and esters like cellulose acetate, an intermediate coating about the core material comprising material that has the affinity, or efficiently binds to another material having the affinity, to bind blood-borne adhesion components that promote the adhesion of cancer cells (such as fibronectin, fibrin, heparin, laminin, tenascin, vitronectin, or their fragments), such as gelatin, collagens, fibrin, dextran and hyaluronate, and blood-borne adhesion components of natural or synthetic origin.

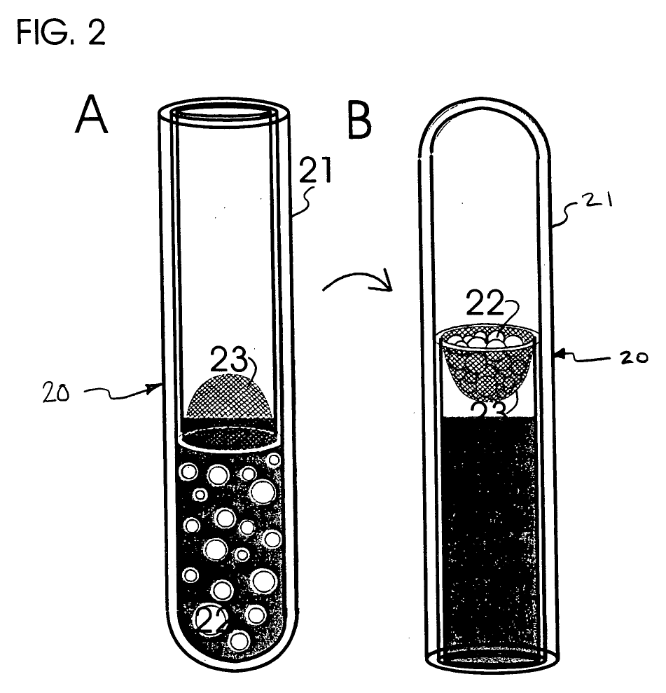

Referring to FIG. 2A there is shown a general schematic of a cell separation system (20) incorporating cell separation beads (22) in a vacuum blood collection tube (21). As seen in FIG. 2A, a conventional mesh filter (23) is incorporated in a blood collection tube to facilitate the washing and the collection of cell-bound beads. Alternatively, the mesh filter (23) can be an independent unit outside the tube. In this case, after incubation of the blood-bead mixture, the mixture can pull into the mesh filter (23) outside the tube to facilitate the washing and the isolation of viable cancer cells from whole blood of patients with metastatic diseases. As seen in FIG. 2B, when incorporated into a vacuum blood collection tube (21) the mesh filter (23) collects the beads.

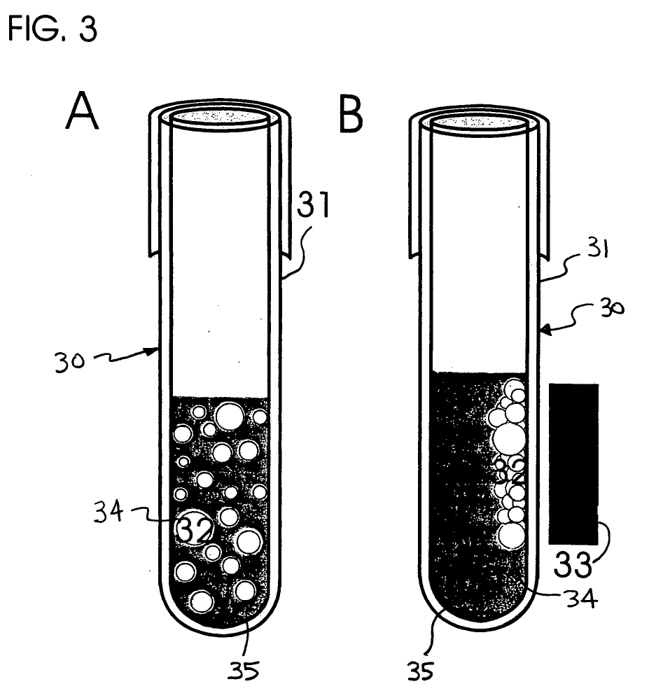

Referring to FIGS. 3A and 3B there is shown a general schematic of a cell separation system (30) comprising a vacuum blood collection tube (31) incorporating microbeads or nanoparticles (32), preferably having a diameter in the range of 20 nm to 20 microns, having an intermediate magnetic coating (34) which is attracted to a magnetic source (33). The beads (32) are typically suspended in blood (35) containing an anticoagulant such as lithium heparin. After incubation of the blood-bead mixture in the tube on shaker with slow speed at 37° C. for 30 minutes to 2 hours, the sample tube is passed through a magnetic field using a magnetic separator (33) to magnetically immobilize microbeads or nanoparticles in the sample having cells bound thereto. This provides a gentle means of washing and collection of cell-bound microbeads or nanoparticles. The microbeads or nanoparticles can capture viable cancer cells and related tissue cells.

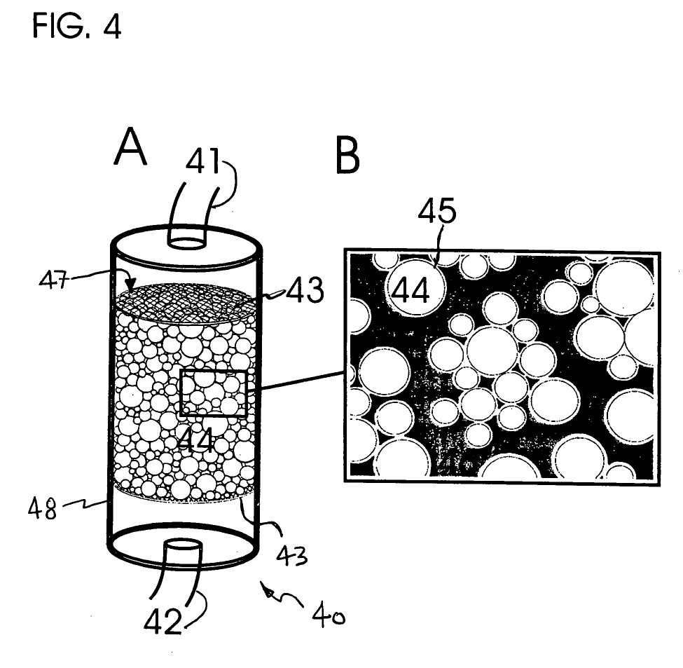

Referring to FIG. 4A there is shown a general schematic of a cell filtration system (40) comprising a filter housing (48) having an inlet (41) in the housing for the introduction of the blood (46) to be filtered; an outlet (42) in the housing for the removal of filtered blood and to return to a patient; and a filter element (47) disposed within the housing, which element comprises a plurality of beads (44), preferably having a diameter in the range of 200 microns to 1,000 microns, which beads are packed tightly between two layers of meshes (43), preferably having mesh opening widths of 50 to 200 microns. The core beads are held back by the meshes. Thus, the filter system allows it to be back-washed with wash liquid.

FIG. 4B is an expanded view of the portion of cell separation beads designated in FIG. 4A, depicting the anastomosic channels formed by the cell separation beads within the inner confinement area and the size and nature of the core bead (44) to be employed. Core beads (44) are coated with an intermediate coating (45) (such as gelatin, collagens, fibrin, hyaluronates and dextran) comprising material that has the affinity, or efficiently binds to another material having the affinity, to bind blood-borne adhesion components that promote the adhesion of cancer cells (such as fibronectin, fibrin, heparin, laminin, tenascin, vitronectin, or their fragments) as well as to bind to the core bead substrate.

In a blood filter, the core material selected must be inert and compatible with the blood, and should be somewhat stiff to adhere well to the intermediate coating (45). Typically materials which may be employed in a blood filter would include, but not be limited to: inert polymeric materials, such as polystyrene, polyamide material like nylon, polyester materials, cellulose ethers and esters like cellulose acetate, urethane foam material, DEAE-dextran, as well as other natural and synthetic materials, such as other foam particles, cotton, wool, dacron, rayon, acrylates and the like. The core material is preferably a polyester, such as a 40 mil 3 denier natural polyester, or non-porous polystyrene plastic. Preferably, in a blood filter, the size of the core materials of beads should not typically exceed about 1 mm, or preferably 200 microns to 1,000 microns in diameter. The core bead material is preferably sorted by their sizes. In blood filters, if the beads are too big, they can compact at high flow rates and less channel surface areas, and, therefore, be less efficient. The nature of the core material to be selected is such that the beads may adhere to the intermediate coating, such as gelatin, collagens, fibrin, hyaluronates and dextran, and create a smooth anastomosic channel within the filter for blood flow.

As seen in FIG. 4B, a modified matrix (45) is coated on the surfaces of packed beads (44) and mesh openings to create anastomosic channels of tremendous surface areas for contacting cells in the fluid. The matrix-coated lining of channels selectively remove viable and aggressive cancer cells and related tissue cells from the blood to be filtered. The filter of this invention is sterile, non-toxic and non-leaking of proteins or particles into blood flow.

FIG. 5 in a schematic representation of a method of the present invention providing for the isolation of viable cancer cells and related tissue cells from tissue samples using a combination of the function-affinity cell separation of the invention and immuno-affinity purification. Fig. S is described in detail in EXAMPLE 6.

### REFERENCES

AI-Mehdi, A. B. et al. 2000. Intravascular origin of metastasis from the proliferation of endothelium-attached tumor cells: a new model for metastasis. Nature Medicine. 6, 100-102.
Asahara, T., Murohara, T., Sullivan, A., Silver, M., Van der Zee, R., Li, T., Witzenbichler, B., Schatteman, G., and Isner, J. M. (1997). Isolation of putative progenitor endothelial cells for angiogenesis. Science 275, 964-967.
Beitsch, P. D. and Clifford, E. (2000). Detection of carcinoma cells in the blood of breast cancer patients. American Journal of Surgery 180, 446-448.
Bell, A. W., Ward, M. A., Blackstock, W. P., Freeman, H. N. M., Choudhary, J. S., Lewis, A. P., Chotai, D., FazeI, A., Gushue, J. N., Paiement, J., Palcy, S., Chevet, E., LafreniereRoula, M., Solari, R., Thomas, D. Y., Rowley, A., and Bergeron, J. J. M. (2001). Proteomics Characterization of Abundant Golgi Membrane Proteins. Journal of Biological Chemistry 276, 5152-5165.
Beltinger, C. P. and Debatin, K. M. (1998). A simple combined microdissection and aspiration device for the rapid procurement of single cells from clinical peripheral blood smears. Molecular Pathology 51, 233-236.
Brugger, W., Bross, K. J., Glatt, M., Weber, F., Mertelsmann, R., and Kanz, L. (1994). Mobilization of tumor cells and hematopoietic progenitor cells into peripheral blood of patients with solid tumors [see comments]. Blood 83, 636-640.
Brugger, W., Buhring, H. J., Grunebach, F., VogeI, W., KauI, S., Muller, R., Brummendorf, T. H., Ziegler, B. L., Rappold, I., Brossart, P., Scheding, S., and Kanz, L. (1999). Expression of MUC-1 epitopes on normal bone marrow: implications for the detection of micrometastatic tumor cells. J. Clin. Oncol. 17, 1535-1544.
Brugger, W., Heimfeld, S., Berenson, R. I., Mertelsmann, R., Kanz, and L. (1995). Reconstitution of hematopoiesis after high-dose chemotherapy by autologous progenitor cells generated ex vivo [see comments]. New England Journal of Medicine 333, 283-287.
Campana, D. et al. (1995). Detection of minimal residual disease in acute leukemia: methodologic advances and clinical significance, Blood 85:1416-34.
Chen, W.-T., and S. J. Singer. 1980. Fibronectin is not present in the focal adhesions formed between normal cultured fibroblasts and their substrata. Proc. Nat. Acad. Sci. U.S.A. 77:7318-7322.
Chen, W.-T., J.-M. Chen, S. J. Parsons, and J. T. Parsons. 1985. Local degradation of fibronectin at sites of expression of the transforming gene product pp60src. Nature 316: 156-158.
Chen, W.-T., K. Olden, B. A. Bernard, F. F. Chu. 1984. Expression of transformation-associated protease(s) that degrade fibronectin at cell contact sites. J. Cell Biol. 98:1546-1555.
Chen, W.-T. and Singer, S. J. (1980). Fibronectin is not present in the focal adhesions formed between normal cultured fibroblasts and their substrata. Proc. Natl. Acad. Sci. U.S. A. 77, 7318-7322.
Chen, W.-T., Yeh, Y., and Nakahara, H. (1994). An in vitro cell invasion assay: determination of cell surface proteolytic activity that degrades extracellular matrix. J. Tiss. Cult. Meth. 16, 177-181.
Clark, R. A., Folkvord, J. M., and Wertz, R. L. (1985). Fibronectin, as well as other extracellular matrix proteins, mediate human keratinocyte adherence. Journal of Investigative Dermatology 84, 378-383.
Cote et al., 1991. Prediction of Early Relapse in Patients With Operable Breast Cancer by Detection of Occult Bone Marrow Micrometastases, Journal of Clinical Oncology, vol. 9, No. 10, pp. 1749-1756.
Dicke et al., 1968. The selective elimination of immunologically competent cells from bone marrow and lymphatic cell mixtures,” Transplantation 6:562-570.
Dicke et al., 1970, “Avoidance of acute secondary disease by purification of hemopoietic stem cells with density gradient centrifugation,” Exp. Hematol. 20:126-130.
Durrant et al., 1992, A rapid method for separating tumor infiltrating cells and tumor cells from colorectal tumors” J. Immunol. Meth. 147:57-64. Ellis et al., 1984, “The use of discontinuous Percoll gradients to separate populations of cells from human bone marrow and peripheral blood,” J. of Immunological Methods 66:9-16.
Fidler, I. J. (1973). The relationship of embolic homogeneity, number, size and viability to the incidence of experimental metastasis. European Journal of Cancer (Oxford) 9, 223227.
Folkman, J. (1995). Seminars in Medicine of the Beth Israel Hospital, Boston. Clinical applications of research on angiogenesis. N. England J. Med. 333, 1757-1763.
Giordano, T. I., Shedden, K. A., Schwartz, D. R., Kuick, R., Taylor, J. M. G., Lee, N., Misek, D. E., Greenson, J. K., Kardia, S. L. R., Beer, D. G., Rennert, G., Cho, K. R., Gruber, S. B., Fearon, E. R., and Hanash, S. (2001). Organ-Specific Molecular Classification of Primary Lung, Colon, and Ovarian Adenocarcinomas Using Gene Expression Profiles. Am J Pathol 159, 1231-1238.
Glaves, D., 1983. Correlation between circulating cancer cells and incidence of metastasis. Br. J. Cancer, 48:665-673.
Glaves, D., Huben, R. P., and Weiss, L. (1988). Haematogenous dissemination of cells from human renal adenocarcinomas. British Journal of Cancer 57, 32-35.
Griwatz et al., 1995. An immunological enrichment method for epithelial cells from peripheral blood, Journal of Immunological Methods, vol. 183, pp. 251-265.
Gross et al, 1995. Moody study detecting breast cancer cells in peripheral blood mononuclear cells at frequencies as low as 10(−7). Proc. Natl. Aca. Sci. USA. 92:537.
Gulati, S C et al. (1993) Rationale for purging in autologous stem cell transplantation. Journal of Hematotherapy, 2:467-71.
Herzenberg et al., 1979, Fetal cells in the blood of pregnant women: detection and enrichment by fluorescence-activated cell sorting. Proc. Natl. Aca. Sci. USA 76: 1453-5.
Hough, C. D., Cho, K. R., Zonderman, A. B., Schwartz, D. R., and Morin, P. J. (2001). Coordinately Up-Regulated Genes in Ovarian Cancer. Cancer Research 61, 3869-3876.
Karczewski, D. M., Lema, M. J., and Glaves, D. (1994). The efficiency of an autotransfusion system for tumor cell removal from blood salvaged during cancer surgery. Anesthesia & Analgesia 78, 1131-1135.
Kraeft, S. K., Sutherland, R., Gravelin, L., Hu, G. H., Ferland, L. H., Richardson, P., Elias, A., and Chen, L. B. (2000). Detection and analysis of cancer cells in blood and bone marrow using a rare event imaging system. Clinical Cancer Research 6, 434-442. Liotta, L. A., Kleinerman, J., and Saidel, G. M. (1974). Quantitative relationships of intravascular tumor cells, tumor vessels, and pulmonary metastases following tumor implantation. Cancer Research 34, 997-1004.
Mann, M., Hendrickson, R. C., and Pandey, A. (2001). ANALYSIS OF PROTEINS AND PROTEOMES BY MASS SPECTROMETRY. Annual Review of Biochemistry 70, 437473.
Mueller, S. C., Ghersi, G., Akiyama, S. K., Sang, Q. X., Howard, L., Pineiro-Sanchez, M., Nakahara, H., Yeh, Y., and Chen, W.-T. (1999). A novel protease-docking function of integrin at invadopodia. J. Biol. Chem. 274, 24947-24952.
Pawson, T. and Scott, J. D. (1997). Signaling Through Scaffold, Anchoring, and Adaptor Proteins. Science 278, 2075-2080.
Peck, K., Sher, Y. P., Shih, J. Y., Roffler, S. R., Wu, C. W., and Yang, P. C. (1998). Detection and quantitation of circulating cancer cells in the peripheral blood of lung cancer patients. Cancer Res. 58, 2761-2765.
Racila, E., Euhus, D., Weiss, A. J., Rao, C., McConneII, J., Terstappen, L. W., and Uhr, J. W. (1998). Detection and characterization of carcinoma cells in the blood. Proceedings of the National Academy of Sciences of the United States of America 95, 4589-4594.
Sabile, A., Louha, M., Bonte, E., Poussin, K., Vona, G., Mejean, A., Chretien, Y., Bougas, L., Lacour, B., Capron, F., Roseto, A., Brechot, C., and Paterlini-Brechot, P. (1999). Efficiency of Ber-EP4 antibody for isolating circulating epithelial tumor cells before RT-PCR detection. American Journal of Clinical Pathology 112, 171-178.
SU, A. I., WelSh, J. B., Sapinoso, L. M., Kern, S. G., Dimitrov, P., Lapp, H., Schultz, P. G., PoweII, S. M., Moskaluk, C. A., Frierson, H. F., Jr., and Hampton, G. M. (2001). Molecular Classification of Human Carcinomas by Use of Gene Expression Signatures. Cancer Research 61, 7388-7393.
Suarez-Quian, C. A., Goldstein, S. R., Pohida, T., Smith, P. D., Peterson, J. I., Wellner, E, Ghany, M., and Bonner, R. F. (1999). Laser capture microdissection of single cells from complex tissues. BioTechniques 26, 328-335.
Ts'o, P. O., Pannek, J., Wang, Z. P., Lesko, S. A., Bova, G. S., Partin, and AW. (1997). Detection of intact prostate cancer cells in the blood of men with prostate cancer. Urology 49, 881-885.
Vona, G., Sabile, A., Louha, M., Sitruk, V., Romana, S., Schutze, K., Capron, F., Franco, D., Pazzagli, M., Vekemans, M., Lacour, B., Brechot, C., and Paterlini-Brechot, P. (2000). Isolation by Size of Epithelial Tumor Cells: A New Method for the Immunomorphological and Molecular Characterization of Circulating Tumor Cells. Am J Pathol 156, 57-63.
Wang, Z. P., Eisenberger, M. A., Carducci, M. A., Partin, A. W., Scher, H I, and Ts'o, P. O. (2000). Identification and characterization of circulating prostate carcinoma cells. Cancer 88, 2787-2795.
Weiss, L. and Glaves, D. (1983). Cancer cell damage at the vascular endothelium. [Review] [62 refs]. Annals of the New York Academy of Sciences 416, 681-692.
Welsh, J. B., Zarrinkar, P. P., Sapinoso, L. M., Kern, S. G., Behling, C. A., Monk, B. J., Lockhart, D. J., Burger, R. A., and Hampton, G. M. (2001). Analysis of gene expression profiles in normal and neoplastic ovarian tissue samples identifies candidate molecular markers of epithelial ovarian cancer. PNAS 98, 1176-1181.

1. A matrix for detecting the presence of cancer cells in a subject, said matrix comprising a solid core material bearing a coating having binding affinity for said solid core material and binding affinity for blood-borne adhesion components that bind cancer cells, and one or more blood-borne adhesion components.

2. The matrix of claim 1 wherein said blood-borne adhesion component is selected from the plasma group consisting of: fibronectin, fibrin, heparin, laminin, tenascin, vitronectin, and biologically functional mimics of the same.

3. The matrix of claim 1 wherein said solid core material is selected from the group consisting of: bone, connective tissue, collagens, gelatin, hyaluronates, fibrin, cotton, wool, polymeric material, polystyrene, magnetic colloid, glass, polyamides, polyesters, cellulose acetate, urethane, DEAE-dextran, dacron, rayon, and acrylate.

4. The matrix of claim 1 wherein said coating comprises an attachment agent having a binding affinity for at least one of the blood-borne adhesion components.

5. The matrix of claim 1 wherein said coating is selected from the group consisting of: gelatin, glutaraldehyde, and gelatin crosslinked with glutaraldehyde;

6. A metastatic cancer cell separation system comprising:
a sealable container having an outer surface and an inner surface;
an adhesion binding material coated on said inner surface of said sealable container, said adhesion binding material having the ability to bind said inner surface of said sealable container and the ability to bind one or more natural or synthetic molecules that have a binding affinity for metastatic cancer cells; and
one or more natural or synthetic molecules having a binding affinity for metastatic cancer cells.

7. The cell separation system of claim 6 wherein said sealable container is a collection tube.

8. The cell separation system of claim 6 wherein said adhesion binding material comprises an attachment agent having a binding affinity for at least one of the blood-borne adhesion components.

9. The cell separation system of claim 6 wherein said adhesion binding material is selected from the group consisting of: gelatin, glutaraldehyde, and gelatin crosslinked with glutaraldehyde.

10. The cell separation system of claim 6 wherein said adhesion binding material further comprises a core material selected from the group consisting of: bone, connective tissue, collagens, gelatin, hyaluronates, fibrin, cotton, wool, polymeric material, polystyrene, magnetic colloid, glass, polyamides, polyesters, cellulose acetate, urethane, DEAE-dextran, dacron, rayon, and acrylate.

11. The cell separation system of claim 6 wherein matrix of claim 1 wherein said one or more natural or synthetic molecules having a binding affinity for metastatic cancer cells is selected from the plasma group consisting of: fibronectin, fibrin, heparin, laminin, tenascin, vitronectin, and biologically functional mimics of the same.

12. A metastatic cancer cell separation system comprising:
a sealable container having an outer surface and an inner surface, said inner surface defining a void;
a plurality of beads coated with an adhesion binding material bound to one or more natural or synthetic molecules that have a binding affinity for metastatic cancer cells, said beads residing within said void;
a separation member positioned in said void in such a manner as to divide said void into two or more compartments said filter having pores of a size to permit filtration of said beads.

13. The cell separation system of claim 12 wherein said sealable container is a collection tube.

14. The cell separation system of claim 13 wherein said adhesion binding material comprises an attachment agent having a binding affinity for at least one of the blood-borne adhesion components.

15. The cell separation system of claim 13 wherein said adhesion binding material is selected from the group consisting of: gelatin, glutaraldehyde, and gelatin crosslinked with glutaraldehyde.

16. The cell separation system of claim 12 wherein said adhesion binding material further comprises a core material selected from the group consisting of: bone, connective tissue, collagens, gelatin, hyaluronates, fibrin, cotton, wool, polymeric material, polystyrene, magnetic colloid, glass, polyamides, polyesters, cellulose acetate, urethane, DEAE-dextran, dacron, rayon, and acrylate.

17. The cell separation system of claim 12 wherein said one or more natural or synthetic molecules having a binding affinity for metastatic cancer cells is selected from the plasma group consisting of: fibronectin, fibrin, heparin, laminin, tenascin, vitronectin, and biologically functional mimics of the same.

18. The cell separation system of claim 12 wherein said cell separation member is a screen.

19. The cell separation system of claim 12 wherein said cell separation member is a filter.

20. A metastatic cancer cell separation system comprising:
a sealable container having an outer surface and an inner surface, said inner surface defining a void;
a plurality of magnetically-attractable microbeads or nanoparticles coated with an adhesion binding material bound to one or more natural or synthetic molecules that have a binding affinity for metastatic cancer cells, said microbeads or nanoparticles residing within said void; and
a magnet on the outer surface of said sealable container, or within said void, of sufficient strength to attract said plurality of magnetic-attractable microbeads or nanoparticles to one location.

21. The cell separation system of claim 20 wherein said sealable container is a collection tube.

22. The cell separation system of claim 20 wherein said sealable container is a flow chamber.

23. The cell separation system of claim 20 wherein said adhesion binding material comprises an attachment agent having a binding affinity for at least one of the blood-borne adhesion components.

24. The cell separation system of claim 20 wherein said adhesion binding material is selected from the group consisting of: gelatin, glutaraldehyde, and gelatin crosslinked with glutaraldehyde.

25. The cell separation system of claim 20 wherein said adhesion binding material further comprises a magnetic colloid intermediate layer in the core material selected from the group consisting of: bone, connective tissue, collagens, gelatin, hyaluronates, fibrin, cotton, wool, polymeric material, polystyrene, glass, polyamides, polyesters, cellulose acetate, urethane, DEAE-dextran, dacron, rayon, and acrylate.

26. The cell separation system of claim 20 wherein said one or more natural or synthetic molecules having a binding affinity for metastatic cancer cells is selected from the plasma group consisting of: fibronectin, fibrin, heparin, laminin, tenascin, vitronectin, and biologically functional mimics of the same.

27. A metastatic cancer cell separation system comprising:
an enclosed container defining a void, said enclosed container having an inlet and an outlet;
a first separation member positioned proximal to said inlet within said void and dividing said void into compartments, said first separation member permitting the flow of at least a component of whole blood therethrough;
a second separation member positioned proximal to said outlet within said void and dividing said void into compartments, said second separation member permitting the flow of at least a component of whole blood therethrough and being positioned antepodal to said first separation member in said void;
a plurality of beads coated with an adhesion binding material bound to one or more natural or synthetic molecules that have a binding affinity for metastatic cancer cells, said beads residing between said first separation member and second separation member and being retained thereby within said void.

28. The cell separation system of claim 27 wherein said adhesion binding material comprises an attachment agent having a binding affinity for at least one of the blood-borne adhesion components.

29. The cell separation system of claim 27 wherein said adhesion binding material is selected from the group consisting of:
gelatin, glutaraldehyde, and gelatin crosslinked with glutaraldehyde.

30. The cell separation system of claim 27 wherein said adhesion binding material further comprises a core material selected from the group consisting of: bone, connective tissue, collagens, gelatin, hyaluronates, fibrin, cotton, wool, polymeric material, polystyrene, magnetic colloid, glass, polyamides, polyesters, cellulose acetate, urethane, DEAE-dextran, dacron, rayon, and acrylate.

31. The cell separation system of claim 27 wherein said one or more natural or synthetic molecules having a binding affinity for metastatic cancer cells is selected from the plasma group consisting of: fibronectin, fibrin, heparin, laminin, tenascin, vitronectin, and biologically functional mimics of the same.

32. The cell separation system of claim 27 wherein said cell separation member is a screen.

33. The cell separation system of claim 27 wherein said cell separation member is a filter.

34. The cell separation system of claim 27 wherein said cell separation beads form the filter unit.
